(19) United States
(12) Patent Application Publication
(10) Pub. No.: US 2025/0064935 A1
(43) Pub. Date: Feb. 27, 2025

(54) NATURAL KILLER CELLS AND CD123 NK CELL ENGAGER COMBINATION THERAPY

(71) Applicant: SANOFI, Paris (FR)

(72) Inventors: Giovanni ABBADESSA, Cambridge, MA (US); Marielle CHIRON, Paris (FR); Alain FOURNIER, Paris (FR); Paula FRAENKEL, Cambridge, MA (US); Angela VIRONE-ODDOS, Paris (FR)

(21) Appl. No.: 18/771,699

(22) Filed: Jul. 12, 2024

Related U.S. Application Data
(60) Provisional application No. 63/526,554, filed on Jul. 13, 2023.

(51) Int. Cl. A61K 39/00; C07K 16/28; C12N 5/0783

(52) U.S. Cl. CPC A61K 39/464419; A61K 39/4613; C07K 16/2866; C12N 5/0646; C12N 2501/2302; C12N 2501/2321; C12N 2501/25

(57) ABSTRACT

Provided herein are compositions and methods of treating or preventing a hematological disease or disorder in a subject in need thereof, comprising expanded NK cells and a binding protein comprising a first antigen binding domain with binding specificity to CD123 and a second antigen binding domain with binding specificity to NKp46.

### CROSS-REFERENCE TO RELATED APPLICATIONS

This application claims priority to U.S. Provisional Patent Application Ser. No. 63/526,554, filed Jul. 13, 2023, the contents of which is hereby incorporated by reference.

### SEQUENCE LISTING

The instant application contains a Sequence Listing which has been submitted electronically in XML format and is hereby incorporated by reference in its entirety. Said XML file, created on Nov. 11, 2024, is named 756090_SA9-381_ST26.xml and is 112,389 bytes in size.

### BACKGROUND

Acute myeloid leukemia (AML), B cell acute lymphoblastic leukemia (B-ALL), and myelodysplastic syndromes (MDS) are heterogeneous clonal neoplastic disease, which are thought to arise from subpopulations of leukemic stem cells, which tend to be resistant to convention chemotherapy, and which may be further responsible for disease relapse.

Natural killer (NK) cells are a subpopulation of lymphocytes that are involved in non-conventional immunity. NK cells provide an efficient immunosurveillance mechanism by which undesired cells such as tumor- or virally-infected cells can be eliminated. Characteristics and biological properties of NK cells include the expression of surface antigens including CD16, CD56 and/or CD57, the absence of the α/β or γ/δ TCR complex on the cell surface, the ability to bind to and kill cells in a MHC-unrestrictive manner and in particular cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate the immune response.

Hematopoietic stem cell transplantation (HSCT) from healthy donors has improved long term survival in patients with hematological diseases and disorders (e.g., AML). However, HSCT comes with limitations, such as the potential for developing graft versus host disease (GvHD), graft failure, and relapse. Additionally, HSCT can come at a financial, mental, and health burden. To overcome the issues with GvHD, NK cells offer an alternative to other immune cells, such as T-cells. NK cell infusions in both preclinical and clinical settings have demonstrated to be effective and can lead to complete remission without GvHD.

Thus, there still is an urgent need for compositions and methods of treatment for patients who are ineligible for or have exhausted standard therapeutic options that also have a greater safety profile than known treatments.

### SUMMARY

In an aspect, provided herein is a composition comprising a Natural killer (NK) cell and a binding protein comprising a first antigen binding domain with binding specificity to CD123 and a second antigen binding domain with binding specificity to NKp46.

In some embodiments, the NK cell is from an autologous donor source. In some embodiments, the NK cell is from an allogeneic donor source.

In some embodiments, the NK cell is expanded in vitro. In some embodiments, the NK cell is expanded in vivo. In some embodiments, the NK cell is isolated form a donor sample prior to being expanded.

In some embodiments, the NK cell is expanded in the presence of other donor sample cells (e.g., PBMCs).

In some embodiments, the NK cell is expanded by contact with a PM particle. In some embodiments, the PM particle comprises a membrane-bound interleukin-21 (IL-21) molecule and a 4-1BBL molecule (e.g., a PM21 particle).

In some embodiments, the composition further comprises IL-12 for the expansion of the NK cell.

In some embodiments, the first antigen binding domain with binding specificity to CD123 comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the a. the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or
b. the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, the VH1 comprises an amino acid sequence of SEQ ID NO: 41, and wherein the VL1 comprises an amino acid sequence of SEQ ID NO: 43; or VH1 comprises an amino acid sequence of SEQ ID NO: 42, and wherein the VL1 comprises an amino acid sequence of SEQ ID NO: 44.

In some embodiments, the second antigen binding domain with binding specificity to NKp46 comprises:
a. a second heavy chain variable domain (VH2) comprising a CDR-H1, H2, and H3 corresponding to the amino acid sequences of:
   i. SEQ ID NO: 13, 14, and 15, respectively;
   ii. SEQ ID NO: 16, 17, and 18, respectively;
   iii. SEQ ID NO: 19, 20, and 21, respectively;
   iv. SEQ ID NO: 22, 23, and 24, respectively; or
   v. SEQ ID NO: 16, 25, and 26, respectively; and
b. a second light chain variable domain (VL2) comprising a CDR-L1, L2, and L3 corresponding to the amino acid sequences of:
   i. SEQ ID NO: 27, 28, and 29, respectively;
   ii. SEQ ID NO: 30, 31, and 32, respectively;
   iii. SEQ ID NO: 33, 34, and 35, respectively;
   iv. SEQ ID NO: 36, 37, and 38, respectively; or
   v. SEQ ID NO: 39, 31, and 40, respectively.

In some embodiments, the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54; the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55; the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56; the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57; the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58; the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

In some embodiments, the VH2 comprises an amino acid sequence of SEQ ID NO: 45, and wherein the VL1 comprises an amino acid sequence of SEQ ID NO: 53; the VH2 comprises an amino acid sequence of SEQ ID NO: 46, and wherein the VL2 comprises an amino acid sequence of SEQ ID NO: 54; the VH2 comprises an amino acid sequence of SEQ ID NO: 47, and wherein the VL2 comprises an amino acid sequence of SEQ ID NO: 55; the VH2 comprises an amino acid sequence of SEQ ID NO: 48, and wherein the VL2 comprises an amino acid sequence of SEQ ID NO: 56; the VH2 comprises an amino acid sequence of SEQ ID NO: 49, and wherein the VL2 comprises an amino acid sequence of SEQ ID NO: 57; the VH2 comprises an amino acid sequence of SEQ ID NO: 50, and wherein the VL2 comprises an amino acid sequence of SEQ ID NO: 58; the VH2 comprises an amino acid sequence of SEQ ID NO: 51, and wherein the VL2 comprises an amino acid sequence of SEQ ID NO: 59; or the VH2 comprises an amino acid sequence of SEQ ID NO: 52, and wherein the VL2 comprises an amino acid sequence of SEQ ID NO: 60.

In some embodiments, the binding protein comprises three polypeptide chains (I), (II) and (III) that form two ABDs, as defined as:

V_{1A}—C_{1A}-Hinge₁-(C_{H}2-C_{H}3)_{A} (I)

V_{1B}—C_{1B}-Hinge₂-(C_{H}2-C_{H}3)_{B}-L₁-V_{2A}-C_{2A}-Hinge₃ (II)

V_{2B}-C_{2B} (III)

wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
   Hinge₁, Hinge₂ and Hinge₃ are identical or different and correspond to all or part of an immunoglobulin hinge region;
   (C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
   L₁ is an amino acid linker.

In some embodiments:
C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO:75;
Hinge₃ corresponds to the amino acid sequence of SEQ ID NO: 77;
L₁ corresponds to the amino acid sequence of SEQ ID NO: 76.

In some embodiments, the residue N297 of the Fc region or variant thereof according to EU numbering comprises a N-linked glycosylation. In some embodiments, the all or part of the Fc region or variant thereof binds to a human CD16A (FcγRIII) polypeptide. In some embodiments, at least two polypeptide chains are linked by at least one disulfide bridge.

In some embodiments, the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

In some embodiments, the V_{1A} is V_{L1} and V_{1B} is V_{H1}. In some embodiments, the V_{2A} is V_{H2} and V_{2B} is V_{L2}.

In some embodiments, V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} Comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} Comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} Comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L}2 comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, (a) V_{H1} and V_{L1} corresponds to the amino acid sequences of SEQ ID NO: 41 and 43 respectively or corresponds to the amino acid sequences of SEQ ID NO: 42 and 44 respectively;

and/or
(b) V_{H2} and V_{L2} corresponds to
   the amino acid sequences of SEQ ID NO: 45 and 53 respectively;
   the amino acid sequences of SEQ ID NO: 46 and 54 respectively;
   the amino acid sequences of SEQ ID NO: 47 and 55 respectively;
   the amino acid sequences of SEQ ID NO: 48 and 56 respectively;
   the amino acid sequences of SEQ ID NO: 49 and 57 respectively;
   the amino acid sequences of SEQ ID NO: 50 and 58 respectively;
   the amino acid sequences of SEQ ID NO: 51 and 59 respectively; or
   the amino acid sequences of SEQ ID NO: 52 and 60 respectively.

In some embodiments,
(a) V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53;
(b) V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L}2 comprises the amino acid sequence of SEQ ID NO: 54;
(c) V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55;
   (d) V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56;
   (e) V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57;
   (f) V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58;
   (g) V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59;
   (h) V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60;
   (i) V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53;
   (j) V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L}2 comprises the amino acid sequence of SEQ ID NO: 54;
   (k) V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55;
   (l) V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56;
   (m) V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57;
   (n) V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58;
   (o) V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59; or
   (p) V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60.

In some embodiments, polypeptide (I) comprises an amino acid sequence of SEQ ID NO: 64; polypeptide (II) comprises an amino acid sequence of SEQ ID NO: 65; and polypeptide (III) comprises an amino acid sequence of SEQ ID NO: 66.

In some embodiments, polypeptide (I) consists of an amino acid sequence of SEQ ID NO: 64; polypeptide (II) consists of an amino acid sequence of SEQ ID NO: 65; and polypeptide (III) consists of an amino acid sequence of SEQ ID NO: 66.

In another aspect, provided herein is a method of treating or preventing a hematological disease or disorder in a subject in need thereof, the method comprising administering a natural killer (NK) cell and a binding protein comprising a first antigen binding domain with binding specificity to CD123 and a second antigen binding domain with binding specificity to NKp46.

In another aspect, provided herein is a method of treating or preventing a hematological disease or disorder in a subject in need thereof, the method comprising administering to the subject a binding protein comprising a first antigen binding domain with binding specificity to CD123 and a second antigen binding domain with binding specificity to NKp46, wherein the first antigen binding domain comprises:
a heavy chain variable domain (V_{H1}) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (V_{L1}) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the NK cell is from an autologous donor source. In some embodiments, the NK cell is from an allogeneic donor source.

In some embodiments, the NK cell is expanded in vitro. In some embodiments, the NK cell is expanded in vivo.

In some embodiments, the NK cell is isolated from a donor sample prior to being expanded. In some embodiments, the NK cell is expanded in the presence of other donor sample cells (e.g., PMBCs).

In some embodiments, the NK cell is expanded by contact with a PM particle. In some embodiments, the PM particle comprises a membrane-bound interleukin-21 (IL-21) molecule and a 4-1BBL molecule (e.g., a PM21 particle). In some embodiments, the method further comprises IL-12 for the expansion of the NK cell.

In some embodiments, the NK cell and the binding protein are administered to the subject intravenously, subcutaneously, intraperitoneally, or intramuscularly. In some embodiments, the NK cell and the binding protein are administered to the subject intravenously.

In some embodiments, the NK cell and the binding protein are administered at different times. In some embodiments, the NK cell is administered prior to administration of the binding protein. In some embodiments, the NK cell is administered after administration of the binding protein. In some embodiments, the NK cell and the binding protein are administered at the same time.

In some embodiments, the first antigen binding domain with binding specificity to CD123 comprises a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, the VH1 comprises an amino acid sequence of SEQ ID NO: 41, and wherein the VL1 comprises an amino acid sequence of SEQ ID NO: 43; or the VH1 comprises an amino acid sequence of SEQ ID NO: 42, and wherein the VL1 comprises an amino acid sequence of SEQ ID NO: 44.

In some embodiments, the second antigen binding domain with binding specificity to NKp46 comprises:
a. a second heavy chain variable domain (VH2) comprising a CDR-H1, H2, and H3 corresponding to the amino acid sequences of:
   i. SEQ ID NO: 13, 14, and 15, respectively;
   ii. SEQ ID NO: 16, 17, and 18, respectively;
   iii. SEQ ID NO: 19, 20, and 21, respectively;
   iv. SEQ ID NO: 22, 23, and 24, respectively; or
   v. SEQ ID NO: 16, 25, and 26, respectively; and
b. a second light chain variable domain (VL2) comprising a CDR-L1, L2, and L3 corresponding to the amino acid sequences of:
   i. SEQ ID NO: 27, 28, and 29, respectively;
   ii. SEQ ID NO: 30, 31, and 32, respectively;
   iii. SEQ ID NO: 33, 34, and 35, respectively;
   iv. SEQ ID NO: 36, 37, and 38, respectively; or
   v. SEQ ID NO: 39, 31, and 40, respectively.

In some embodiments, the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54; the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55; the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56; the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57; the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58; the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

In some embodiments, the VH2 comprises an amino acid sequence of SEQ ID NO: 45, and wherein the VL1 comprises an amino acid sequence of SEQ ID NO: 53; the VH2 comprises an amino acid sequence of SEQ ID NO: 46, and wherein the VL2 comprises an amino acid sequence of SEQ ID NO: 54; the VH2 comprises an amino acid sequence of SEQ ID NO: 47, and wherein the VL2 comprises an amino acid sequence of SEQ ID NO: 55; the VH2 comprises an amino acid sequence of SEQ ID NO: 48, and wherein the VL2 comprises an amino acid sequence of SEQ ID NO: 56; the VH2 comprises an amino acid sequence of SEQ ID NO: 49, and wherein the VL2 comprises an amino acid sequence of SEQ ID NO: 57; the VH2 comprises an amino acid sequence of SEQ ID NO: 50, and wherein the VL2 comprises an amino acid sequence of SEQ ID NO: 58; the VH2 comprises an amino acid sequence of SEQ ID NO: 51, and wherein the VL2 comprises an amino acid sequence of SEQ ID NO: 59; or the VH2 comprises an amino acid sequence of SEQ ID NO: 52, and wherein the VL2 comprises an amino acid sequence of SEQ ID NO: 60.

In some embodiments, the binding protein comprises three polypeptide chains (I), (II) and (III) that form two ABDs, as defined below:

V_{1A}—C_{1A}-Hinge₁-(C_{H}2-C_{H}3)_{A} (I)

V_{1B}—C_{1B}-Hinge₂-(C_{H}2-C_{H}3)_{B}-L₁-V_{2A}-C_{2A}-Hinge₃ (II)

V_{2B}—C_{2B} (III)

wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
   Hinge₁, Hinge₂ and Hinge₃ are identical or different and correspond to all or part of an immunoglobulin hinge region;
   (C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
   L₁ is an amino acid linker.

In some embodiments, C_{1B} is an immunoglobulin heavy chain constant domain 1 (C_{H}1); C_{2A} is an immunoglobulin heavy chain constant domain 1 (C_{H}1); C_{L} corresponds to an immunoglobulin kappa light chain constant domain (C_{κ}); (C_{H}2-C_{H}3)_{A} corresponds to the amino acid sequence of SEQ ID NO: 69; (C_{H}2-C_{H}3)_{B} corresponds to the amino acid sequence of SEQ ID NO: 70; Hinge₁ corresponds to the amino acid sequence of SEQ ID NO:74; Hinge₂ corresponds to the amino acid sequence of SEQ ID NO:75; Hinge₃ corresponds to the amino acid sequence of SEQ ID NO: 77; and L₁ corresponds to the amino acid sequence of SEQ ID NO: 76.

In some embodiments, residue N297 of the Fc region or variant thereof according to EU numbering comprises a N-linked glycosylation. In some embodiments, all or part of the Fc region or variant thereof binds to a human CD16A (FcγRIII) polypeptide. In some embodiments, at least two polypeptide chains linked by at least one disulfide bridge.

In some embodiments, the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

In some embodiments, V_{1A} is V_{L1} and V_{1B} is V_{H1}. In some embodiments, V_{2A} is V_{H2} and V_{2B} is V_{L2}.

In some embodiments, V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} Comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} Comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} Comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, (a) V_{H1} and V_{L1} corresponds to the amino acid sequences of SEQ ID NO: 41 and 43 respectively or corresponds to the amino acid sequences of SEQ ID NO: 42 and 44 respectively; and/or (b) V_{H2} and V_{L2} corresponds to the amino acid sequences of SEQ ID NO: 45 and 53 respectively; the amino acid sequences of SEQ ID NO: 46 and 54 respectively; the amino acid sequences of SEQ ID NO: 47 and 55 respectively; the amino acid sequences of SEQ ID NO: 48 and 56 respectively; the amino acid sequences of SEQ ID NO: 49 and 57 respectively; the amino acid sequences of SEQ ID NO: 50 and 58 respectively; the amino acid sequences of SEQ ID NO: 51 and 59 respectively; or the amino acid sequences of SEQ ID NO: 52 and 60 respectively.

In some embodiments, V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53; VH1 comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54; V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55; V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56; V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57; V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58; V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59; V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60; VH1 comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53; V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54; V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55; V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56; V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57; V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58; V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59; or V_{H1} comprises the amino acid sequence of SEQ ID NO: 42; V_{L1} Comprises the amino acid sequence of SEQ ID NO: 44; V_{H2} comprises the amino acid sequence of SEQ ID NO: 52; VL2 comprises the amino acid sequence of SEQ ID NO: 60.

In some embodiments, polypeptide (I) comprises an amino acid sequence of SEQ ID NO: 64; polypeptide (II) comprises an amino acid sequence of SEQ ID NO: 65; and polypeptide (III) comprises an amino acid sequence of SEQ ID NO: 66.

In some embodiments, polypeptide (I) consists of an amino acid sequence of SEQ ID NO: 64; polypeptide (II) consists of an amino acid sequence of SEQ ID NO: 65; and polypeptide (III) consists of an amino acid sequence of SEQ ID NO: 66.

In some embodiments the hematological disease or disorder is a leukemia. In some embodiments, the leukemia is acute myeloid leukemia (AML). In some embodiments, the AML is relapsed or refractory.

In another aspect, provided herein is a method of treating or preventing a hematological disease or disorder in a subject in need thereof, the method comprising administering to the subject a Natural Killer (NK) cell and a binding protein comprising a first antigen binding domain with binding specificity to CD123 and a second antigen binding domain with binding specificity to NKp46,
wherein the first antigen binding domain comprises a heavy chain variable domain (VH1) and a light chain variable domain (VL1), wherein:
   the VH1 comprises a complementary determining region (CDR)-H1, H2 and H3 corresponding to the amino acid sequences of SEQ ID NO: 1, 2, and 3; and
   the VL1 comprises a CDR-L1, L2 and L3 corresponding to the amino acid sequences of SEQ ID NO: 7, 8, and 9; and
wherein the second antigen binding domain comprises a heavy chain variable domain (VH2) and a light chain variable domain (VL2), wherein:
   the VH2 comprises a CDR-H1, H2, and H3 corresponding to the amino acid sequences of SEQ ID NO: 13, 14, and 15; and
   the VL2 comprises a CDR-L1, L2, and L3 corresponding to the amino acid sequences of SEQ ID NO: 27, 28, and 29;
   and wherein all or part of the immunoglobulin Fc region or variant thereof binds to a human Fc-γ receptor.

In another aspect, provided herein is a method of treating or preventing a hematological disease or disorder in a subject in need thereof, the method comprising administering to the subject a Natural Killer (NK) cell and a binding protein comprising a first antigen binding domain with binding specificity to CD123, a second antigen binding domain with binding specificity to NKp46, and all or part of an immunoglobulin Fc region or variant thereof that binds to a human Fc-7 receptor, wherein the binding protein comprises:
a polypeptide (I) that consists of an amino acid sequence of SEQ ID NO: 64;
a polypeptide (II) that consist of an amino acid sequence of SEQ ID NO: 65; and
a polypeptide (III) that consists of an amino acid sequence of SEQ ID NO: 66.

### BRIEF DESCRIPTION OF THE DRAWINGS

If not specified otherwise, the binding proteins of the present disclosure are oriented with the amino terminal direction (“N-terminal end” or “N-term”) on the left-hand side and the carboxyl-terminal direction (“C-terminal end” or “C-term”) on the right-hand side, in accordance with standard usage and convention.

FIG. 1 depicts a tri-dimensional schematic representation of the F25 format which is a variant of the bispecific F5 format, including one human NKp46 binding site and one human CD123 binding site. As depicted in FIG. 1, the C-term of the polypeptide is on the left-hand side and the N-term is on the right-hand side.

FIG. 2A-2D illustrates two-dimensional schematic representations of F25, F5, F26 and F6 formats respectively including the relevant domains for each polypeptide chain. In FIG. 2A to 2D, the C-term of the polypeptide is on the left-hand side and the N-term is on the right-hand side. The human NKp46 binding domain is formed by the VH/VL pair on the left side. The human CD123 binding domain is formed by the VH/VL pair on the right side.

FIG. 2A shows a two-dimensional schematic representation of the F25 format. This representation represents the claimed “NKp46-CD123_F25” binding protein.

FIG. 2B shows a two-dimensional schematic representation of the F5 format. When compared to F25, the F5 differs in that the CL and CH pair of the NKp46 binding domain are swapped, with the third polypeptide chain comprising a CH1 domain and a VL domain.

FIG. 2C shows a two-dimensional schematic representation of the F26 format. This F26 differs from F25 of FIG. 2A in that it includes a Fc-silent N297S mutation on each CH2 domain.

FIG. 2D shows a two-dimensional schematic representation of the F6 format. This F6 differs from F5 of FIG. 2B in that it includes a Fc-silent N297S mutation on each CH2 domain.

FIG. 2E shows a two-dimensional detailed representation of a variant of the F25 format. This F25 format representation corresponds to the one in FIG. 2A.

FIG. 3 shows a proposed mechanism of action of a NK Cell Engager (NKCE) for killing, following joint binding of a tumor cell (i.e. an AML cell line; i.e. MOLM-13) expressing CD123 and a NK cell expressing NKp46 and a Fcγ receptor (CD16a). Reproduced and adapted from Gauthier, L. et al. (“Multifunctional natural killer cell engagers targeting NKp46 trigger protective tumor immunity”. Cell 177, 1701-1713 (2019)).

FIG. 4 shows the cytotoxic activity of the K-NK cells from 6 different donors in combination with CD123 NKCE at different concentrations against THP-1 cells. The effector cell (E) to target cell (T) ratio (E:T) was 1:1. Growth curves of the THP-1 GFP cells alone are depicted in black.

FIG. 5 shows the cytotoxic activity of the K-NK cells from 6 different donors in combination with CD123 NKCE at different concentrations against THP-1 cells. The effector cell (E) to target cell (T) ratio (E:T) was 3:1. Growth curves of the THP-1 GFP cells alone are depicted in black.

FIG. 6 illustrates the difference in NKp46 and CD16 expression after activation of NK cells from peripheral blood with PM21 particles. Following PM21 particle activation, the NK cells (e.g., the K-NK cells) co-express higher levels of NKp46 and CD16 within the CD56bright population, which may lead to more optimal engagement of the CD123 NKCE with the K-NK cells.

### DETAILED DESCRIPTION

This disclosure provides compositions and methods of treating hematological diseases and disorders (e.g., acute myeloid leukemia (AML)) comprising activated NK cells and multifunctional binding proteins that bind one surface biomarker on immune NK cells, i.e., NKp46 and one antigen of interest on tumoral target cells, i.e., CD123, and is capable of redirecting NK cells to lyse a target cell that expresses the CD123 surface biomarker. The multifunctional binding proteins of the present disclosure further comprises all or part of a Fc region or variant thereof which binds a Fc-receptor (FcγR), in particular an activating Fc-γ receptor (FcγR), for example FcγRIIIa also called CD16a.

### I. Definitions

That the disclosure may be more readily understood, select terms are defined below.

The articles “a” and “an” are used herein to refer to one or to more than one (i.e., to at least one) of the grammatical object of the article.

“About” or “approximately” as used herein when referring to a measurable value such as an amount, a temporal duration, and the like, is meant to encompass variations of ±20% or ±10%, ±5%, ±1%, or ±0.1% of a given value or range, as such variations are appropriate to perform the disclosed methods.

As used herein, the term “Cluster of Differentiation 123” or “CD123” marker is also known as “Interleukin 3 receptor alpha (IL3RA)” or “IL3R”, “IL3RX”, “IL3RY”, “IL3RAY”, “hIL-3Ra” and denotes an interleukin 3 specific subunit of a heterodimeric cytokine receptor. The functional interleukin 3 receptor is a heterodimer that comprises a specific alpha chain (IL-3A; CD123) and the IL-3 receptor beta chain (βθ; CD131) that is shared with the receptors for granulocyte macrophage colony stimulating factor (GM-CSF) and interleukin 5 (IL-5). CD123 is a type I integral transmembrane protein with a deduced Molecular Weight of about 43 kDa containing an extracellular domain involved in IL-3 binding, a transmembrane domain and a short cytoplasmic tail of about 50 amino acids. The extracellular domain is composed of two regions: a N-terminal region of about 100 amino acids, the sequence of which exhibits similarity to equivalent regions of the GM-CSF and IL-5 receptor alpha-chains; and a region proximal to the transmembrane domain that contains four conserved cysteine residues and a motif, common to other members of this cytokine receptor family. The IL-3 binding domain comprises about 200 amino acid residue cytokine receptor motifs (CRMs) made up of two Ig-like folding domains. The extracellular domain of CD123 is highly glycosylated, with N-glycosylation necessary for both ligand binding and receptor signaling. The protein family gathers three members: IL3RA (CD123A), CSF2RA and IL5RA. The overall structure is well conserved between the three members, but sequence homologies are very low. One 300 amino-acid long isoform of CD123 has been discovered so far, but only on the RNA level which is accessible on the Getentry database under the accession number ACM241 16.1. A reference sequence of full-length human CD123 protein, including signal peptide, is available from the NCBI database under the accession number NP_002174.1 and under the Uniprot accession number P26951.

The extracellular domain of human CD123 (ECD) consists of the amino acid sequence of SEQ ID NO: 82. CD123 (the interleukin-3 receptor alpha chain IL-3Ra) is a tumor antigen overexpressed in a variety of hematological neoplasms. The majority of AML blasts express surface CD123 and this expression does not vary by subtype of AML. Higher expression of CD123 on AML at diagnosis has been reported to be associated with poorer prognosis.

CD123 expression has been reported in other hematological malignancies including myelodysplasia, systemic mastocytosis, blastic plasmacytoid dendritic cell neoplasm (BPDCN), ALL and hairy cell leukemia.

As used herein, “Natural killer” or “NK cells” refers to a sub-population of lymphocytes that is involved in non-conventional immunity. NK cells can be identified by virtue of certain characteristics and biological properties, such as the expression of specific surface antigens including CD16, CD56 and/or CD57, NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell KS and Colonna M). Human Press. pp. 219-238 (2000).

As used herein, the term “K-NK cell” refers to a NK cell that has been selectively activated and stimulated, either in isolation or in the presence of other peripheral blood mononuclear cells (PBMCs), by PM particles or feeder cell particles (membrane bound IL-21). In some embodiments, the K-NK cells are isolated and identified by particular cellular markers (e.g., CD56+, CD16, NKp46, NKG2D). Prior to administration, the K-NK cells may be assayed for cytokine production in response to a co-culture with target cells (e.g., K562 cells). In some embodiments, the cytokines are IFNγ and/or TNFα.

As used herein, the term “PM particle” refers to a membrane particle used to selectively activate, stimulate, and expand NK cells to produce K-NK cells. PM particles are prepared from the plasma membranes of feeder cells (e.g., K562 cells) that express one or more NK cell effector agents coupled to a membrane-inserting peptide (e.g., Fc, GPI, transmembrane T-cell receptor, or pHLIP) in the plasma membrane. A membrane-inserting peptide may be a molecule that promotes insertion into a membrane. Membrane-inserting peptides may comprise segments of CD4 or an IgG with affinity for a lipid bilayer. The membrane self-inserting peptide may be any peptide known to insert into a cell membrane. Depending on the use of the membrane self-inserting peptide conjugate, certain membrane self-inserting peptides can be better choices than others. One of skill in the art would understand what membrane self-inserting peptide is ideal under different circumstances. In some embodiments, the NK cell effector agent is a cytokine. In some embodiments, the cytokine is a membrane-bound cytokine. In some embodiments, the cytokine is interleukin-21 (IL-21). In some embodiments, the cytokine is interleukin-15 (IL-15). In some embodiments, the NK cell effector agent is a stimulatory peptide. In some embodiments, the stimulatory peptide is 4-1BB ligand (4-1BBL, TFNSF9) or a functional fragment thereof. In some embodiments, the membrane particle comprises membrane-bound IL-15 (“PM15 particle”). In some embodiments, the membrane particle comprises membrane-bound IL-21 and 4-1BBL (“PM21 particle”).

In some embodiments, the plasma membrane PM21 particles can be purified from feeder cells that stimulate NK cells. In some embodiments, the feeder cells can be K562 cells transfected to express membrane-bound IL-21 and 41BBL.

As used herein, the term “NKp46” marker, or “Natural cytotoxicity triggering receptor 1”, also known as “CD335” or “NKP46” or “NK-p46” or “LY94” refers to a protein or polypeptide encoded by the Ncr1 gene. A reference sequence of full-length human NKp46 protein is available from the NCBI database under the accession number NP_004820. The human NKp46 extracellular domain (ECD) corresponds to the amino acid sequence of SEQ ID NO: 80. The human NKp46 mRNA sequence is described in NCBI accession number NM_004829.

As used herein, the term “Fc-γ receptor” or “FcγR” or “Fc-gamma receptor” may refer to both activating and inhibitory FcγRs. Fc-gamma receptors (FcγR) are cellular receptors for the Fc region of an Immunoglobulin G (IgG). Upon binding of complexed IgG, FcγRs can modulate cellular immune effector functions, thereby linking the adaptive and innate immune systems, including ADCC-mediated immune responses. In humans, six classic FcγRs are currently reported: one high-affinity receptor (FcγRI) and five low-to-medium-affinity FcγRs (FcγRIIA, —B and —C, FcγRIIIA and —B). All FcγRs bind the same region on IgG Fc, yet with differing high (FcgRI) and low (FcgRII and FcgRIII) affinities. On a functional level, most of the FcγRs are activating receptors that can induce the cellular responses mentioned above, including ADCC-mediated immune response. Whereas FcγRI, FcγRIIa, FcγRIIc, and FcγRIIIa are activating receptors characterized by an intracellular immunoreceptor tyrosine-based activation motif (ITAM), FcγRIIb has an inhibition motif (ITIM) and is therefore inhibitory. Unless specified otherwise, the term FcγRs encompasses activating receptors, including FcγRI (CD64), FcγRIIA (CD32a), FcγRIIIa (CD16a) and FcγRIIIb (CD16b), and preferably FcγRIIIa (CD16a).

As used herein, the terms “FcγRIIIa (CD16a)” or “FcγRIIIa” or “CD16a” or “CD16” or “Cluster of Differentiation 16” may refer to a 50-65 kDa cell surface molecule expressed on mast cells, macrophages, and natural killer cells as a transmembrane receptor. FcγRIIIa is an activating receptor containing immunoreceptor tyrosine activating motifs (ITAMs) in the associated FcR γ-chain, ITAMs being necessary for receptor expression, surface assembly and signaling. CD16a is a low affinity receptor for IgG and is an important receptor mediating ADCC (antibody dependent cell mediated cytotoxicity) by NK cells. The high affinity receptor CD16a is preferentially found on NK cells and monocytes and induces antibody-dependent cellular cytotoxicity (ADCC) upon IgG binding.

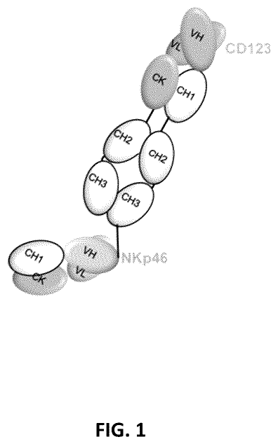

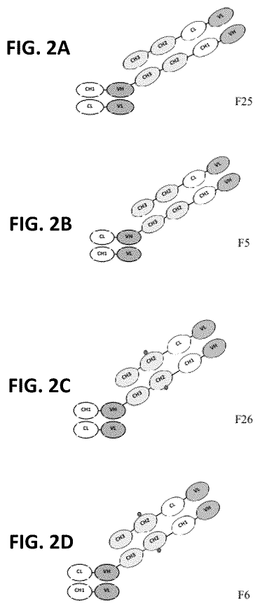

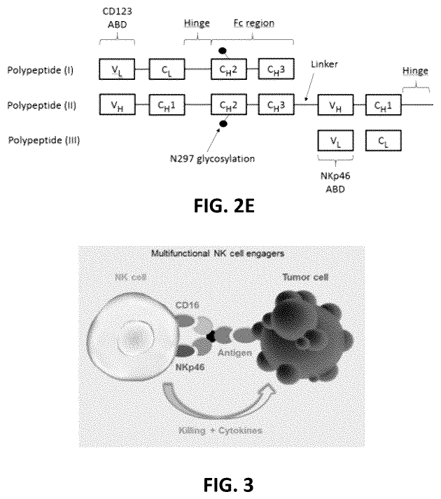

As used herein, the terms “Format 5” or “F5”, “Format 25” or “F25”, “Format F6” or “F6” and “Format 26” or “F26” refer to specific binding protein configurations of bispecific or multispecific antibodies specifically designed to engineer multiple antigen binding domains into a single antibody molecule. The multifunctional binding proteins of the present disclosure which comprise a NKp46-binding domain and a CD123-binding domain, are made based on the F25 format, as exemplified in FIG. 1 and FIG. 2. F25 and format F26 respectively differ from format F5 and F6 in that one CH1/CL pair between the second and third polypeptide chain are swapped to form a CL/CH1 pair. The F5 and F6 format have been previously described in the international patent application WO2017114694, incorporated herein by reference.

As used herein, the term “bispecific binding protein” refers to a binding protein that specifically binds to two different antigen targets (e.g., human NKp46 and human CD123) through at least two distinct antigen-binding domains (ABDs). A bispecific binding protein may be bivalent (two ABDs) or multivalent (more than two ABDs).

As used herein, the terms “specifically binds to” or “binds specifically to” refers to the ability of an antigen-binding domain (ABD) to bind to an antigen (e.g. human NKp46 and/or human CD123) containing an epitope with an Kd of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or to bind to an epitope with an affinity that is at least twofold greater than its affinity for a nonspecific antigen.

As used herein, the term “specifically binds to human NKp46 polypeptide” may refer to a specific binding toward a polypeptide comprising an amino acid sequence of SEQ ID NO: 80.

As used herein, the term “specifically to a human CD123 polypeptide” may refer to a specific binding toward a polypeptide comprising an amino acid sequence of SEQ ID NO: 82.

As used herein, the term “binds to a human Fc-7 receptor polypeptide” may refer to a binding toward a polypeptide comprising an amino acid sequence of SEQ ID NO: 83 or SEQ ID NO: 84.

Competitive binding assays and other methods for determining specific binding are further described below and are well known in the art. Expressions such as “specifically binds to”, or “with specificity for” are used interchangeably. Those terms are not construed to refer exclusively to those antibodies, polypeptides and/or multichain polypeptides which actually bind to the recited target/binding partner, but also to those which, although provided in a non-bound form, retain the specificity to the recited target. Binding specificity can be quantitatively determined by an affinity constant KA (or K_{A}) and a dissociation constant KD (or K_{D}).

As used herein, the term “affinity”, concentration (EC₅₀) or the equilibrium dissociation constant (K_{D}) means the strength of the binding of an antibody or polypeptide to an epitope. The affinity of an antibody is given by a specific type of equilibrium constant, which is the dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant KA is defined by 1/KD. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BIAcore™ SPR analytical device). In a non-limitative manner, a KD of less than 50 nM as determined by SPR, and under physiological conditions (e.g. at a pH ranging from 6 to 8 under normal buffer conditions), may generally be considered as indicative of specificity of binding for antigen-antigen binding domain (ABD) interactions.

As an illustration, and according to some particular and exemplified embodiments, binding proteins reported herein comprise:
an antigen binding domain which binds specifically to human CD123 with a K_{D} of less than 10 nM, in particular with a K_{D} of less than 0.5 nM, as determined by SPR, under physiological conditions;
an antigen binding domain which binds specifically to human NKp46 with a K_{D} of less than 50 nM, in particular with a K_{D} of less than 20 nM, as determined by SPR, under physiological conditions.

As used herein, the term “and/or” is a grammatical conjunction that is to be interpreted as encompassing that one or more of the cases it connects may occur. For example, the wording “such native sequence proteins can be made using standard recombinant and/or synthetic methods” indicates that native sequence proteins can be made using standard recombinant and synthetic methods or native sequence proteins can be made using standard recombinant methods or native sequence proteins can be made using synthetic methods.

As used herein, “treating” refers to a therapeutic use (i.e., on a subject having a given disease) and means reversing, alleviating, inhibiting the progress of one or more symptoms of such disorder or condition. Therefore, treatment does not only refer to a treatment that leads to a complete cure of the disease, but also to treatments that slow down the progression of the disease and/or prolong the survival of the subject.

As used herein, “preventing” means a prophylactic use (i.e., on a subject susceptible of developing a given disease and encompasses the treatment of relapsed AML patient.

As used herein, the terms “therapeutically effective amount” of the multifunctional binding protein or pharmaceutical composition thereof is meant a sufficient amount of the antibody-like multifunctional binding protein to treat said cancer disease, at a reasonable benefit/risk ratio applicable to any medical treatment. It will be understood, however, that the total daily usage of the polypeptides and compositions of the present disclosure will be decided by the attending physician within the scope of sound medical judgment. The specific therapeutically effective dose level for any particular patient will depend upon a variety of factors including the disorder being treated and the severity of the disorder; activity of the specific polypeptide employed; the specific composition employed, the age, body weight, general health, sex and diet of the patient; the time of administration, route of administration, and rate of excretion of the specific polypeptide employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved.

As used herein, the term “subject” or “individual” or “patient” are used interchangeably and may encompass a human or a non-human mammal, rodent or non-rodent. The term includes, but is not limited to, mammals, e.g., humans including man, woman and child, other primates (monkey), pigs, rodents such as mice and rats, rabbits, guinea pigs, hamsters, cows, horses, cats, dogs, sheep and goats.

As used herein, the singular form “a”, “an” and “the” include plural references unless the context clearly dictates otherwise. For example, the term “a pharmaceutically acceptable carrier” encompasses a plurality of pharmaceutically acceptable carriers, including mixtures thereof.

As used herein, “a plurality of” may thus include«two» or «two or more».

As used herein, “antibody” or “immunoglobulin” may refer to a natural or conventional antibody in which two heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain generally includes four domains, a variable domain (VH) and three constant domains (CH1, CH2 and CH3, collectively referred to as CH). In particular, classes IgG, IgA, and IgD have three heavy chain constant region domains, which are designated CH1 CH2, and CH3; and the IgM and IgE classes have four heavy chain constant region domains, CH1, CH2, CH3, and CH4. The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the antigen-binding fragment (Fab) of an immunoglobulin and consists of the variable portions of one light chain and one heavy chain.

As used herein, when referring to “IgG” or “Immunoglobulin G” in general, IgG1, IgG2, IgG3 and IgG4 are included, unless defined otherwise. In particular, IgG is IgG1.

As used herein, the term “antibody-like” or “immunoglobulin-like” polypeptide may also refer to non-conventional or synthetic antigen-binding polypeptides or binding protein, including single domain antibodies and fragments thereof, in particular variable heavy chain of single domain antibodies, and chimeric, humanized, bispecific or multimeric antibodies.

As used herein, the term “multifunctional binding protein” encompass a multi-chain protein, including but not limited to antibody-like polypeptide or protein formats, which comprises at least one first variable region (e.g. a first immunoglobulin heavy chain variable domain (VH) and/or an immunoglobulin light chain variable domain (VL)) binding specifically to a human CD123 polypeptide, and at least one second variable region (e.g. a second immunoglobulin heavy chain variable domain (VH) and/or immunoglobulin light chain variable domain (VL)) binding specifically to a human NKp46 polypeptide. Although not limited specifically to a particular type of construct, one general embodiment is particularly considered throughout the specification: the polypeptide constructs reported in WO2015197593 and WO2017114694, each of which is incorporated herein by reference. In particular, the multifunctional binding protein such as those reported in WO2015197593 and WO2017114694, may encompass any construct comprising one or more polypeptide chains.

As used herein, the term “humanized”, as in “humanized antibody” refers to a polypeptide (i.e., an antibody or an antibody-like polypeptide) which is wholly or partially of non-human origin and which has been modified to replace certain amino acids, in particular in the framework regions of the heavy and light chains, in order to avoid or minimize an immune response in humans. The constant domains of a humanized antibody are most of the time human CH and CL domains. Numerous methods for humanization of an antibody sequence are known in the art; see e.g., the review by Almagro & Fransson (2008) Front Biosci. 13: 1619-1633. One commonly used method is CDR grafting, or antibody reshaping, which involves grafting of the CDR sequences of a donor antibody, generally a mouse antibody, into the framework scaffold of a human antibody of different specificity.

For chimeric antibodies, humanization typically involves modification of the framework regions of the variable region sequences. Amino acid residues that are part of a CDR will typically not be altered in connection with humanization, although in certain cases it may be desirable to alter individual CDR amino acid residues, for example to remove a glycosylation site, a deamidation site or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, in particular by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues. Substitution in a CDR sequence to remove one of the implicated residues is also intended to be encompassed by the claimed multifunctional binding protein.

As used herein, the term “conservative amino acid substitution” refers to substitutions in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine), and aromatic side chains (e.g., tyrosine, phenylalanine, tryptophan). When an amino acid belongs to two different classes (i.e., tyrosine & phenylalanine), both can be accepted. As a reference, the following classification will be followed throughout the specification, unless stated otherwise.

| Conservative Substitution | Type of Amino acid |
|---|---|
| Ala, Val, Leu, Ile, Met, Pro, Phe, | Amino acids with non-polar side |
| Trp | chains |
| Ser, Thr, Tyr, Asn, Gln, Cys, Gly | Amino acids with uncharged polar |
| | side chains |
| Asp, Glu | Amino acids with acidic side chains |
| Lys, Arg, His | Amino acids with basic side chains |
| Tyr, Phe, Trp, | Aromatic amino acids |

As used herein, the term “domain” may be any region of a protein, generally defined on the basis of sequence homologies or identities, which is related to a specific structural or functional entity. Accordingly, the term “region”, as used in the context of the present disclosure, is broader in that it may comprise additional regions beyond the corresponding domain.

As used herein, the terms “linker region”, “linker peptide” or “linker polypeptide” or “amino acid linker” or “linker” refer to any amino acid sequence suitable for covalently linking two polypeptide domains, such as two antigen-binding domains together and/or a Fc region to one or more variable regions, such as one or more antigen-binding domains. Although the term is not limited to a particular size or polypeptide length, such amino acid linkers are generally less than 50 amino acids in length, preferably less than 30 amino acids in length, for instance 20 or less than 20 amino acids in length, for instance 15 or less than 15 amino acids in length. Such amino acid linkers may optionally comprise all or part of an immunoglobulin polypeptide chain, such as all or part of a hinge region of an immunoglobulin. Alternatively, the amino acid linker may comprise a polypeptide sequence that is not derived from a hinge region of an immunoglobulin, or even that is not derived from an immunoglobulin heavy or light polypeptide chain.

As used herein, an immunoglobulin hinge region, or a fragment thereof, may thus be considered as a particular type of linker, which is derived from an immunoglobulin polypeptide chain.

As used herein, the term “hinge region” or “hinge” refers to a generally flexible region and born by the corresponding heavy chain polypeptides, and which separates the Fc and Fab portions of certain isotypes of immunoglobulins, more particularly of the IgG, IgA or IgD isotypes. Such hinge regions are known in the Art to depend upon the isotype of immunoglobulin which is considered. For native IgG, IgA and IgD isotypes, the hinge region thus separates the CH1 domain and the CH2 domain and is generally cleaved upon papain digestion. On the other hand, the region corresponding to the hinge in IgM and IgE heavy chains is generally formed by an additional constant domain with lower flexibility. Additionally, the hinge region may comprise one or more cysteines involved in interchain disulfide bonds. The hinge region may also comprise one or more binding sites to a Fcγ receptor, in addition to FcγR binding sites born by the CH2 domain, when applicable. Additionally, the hinge region may comprise one or more post-translational modification, such as one or more glycosylated residues depending on the isotype which is considered. Thus, it will be readily understood that the reference to the term “hinge” throughout the specification is not limited to a particular set of hinge sequences or to a specific location on the structure. Unless instructed otherwise, the hinge regions which are still particularly considered comprise all or part of a hinge from an immunoglobulin belonging to one isotype selected from: the IgG isotype, the IgA isotype and the IgD isotype; in particular the IgG isotype.

As used herein, the terms “CH domain”, or “CH domain”, or “constant domain”, can be used interchangeably and refer to any one or more heavy chain immunoglobulin constant domain(s). Such CH domains are natively folded as immunoglobulin-like domains, although they may be partly disordered in an isolated form (e.g., CH1 domains when not associated with the constant domain of a light chain (CL)). Unless instructed otherwise, the term may thus refer to a CH1 domain, a CH2 domain, a CH3 domain; or any combinations thereof.

As used herein, the terms “CH1 domain”, or “CH1 domain”, or “constant domain 1”, can be used interchangeably and refer to the corresponding heavy chain immunoglobulin constant domain 1.

As used herein, the term “CH2 domain”, or “CH2 domain”, or “constant domain 2” can be used interchangeably and refer to the corresponding heavy chain immunoglobulin constant domain 2.

As used herein, the term “CH3 domain”, or “CH3 domain”, or “constant domain 3” can be used interchangeably and refer to the corresponding heavy chain immunoglobulin constant domain 3.

As used herein, the term “CH2-CH3”, as in (CH2-CH3)A and (CH2-CH3)B, thus refers to a polypeptide sequence comprising an immunoglobulin heavy chain constant domain 2 (CH2) and an immunoglobulin heavy chain constant domain 3 (CH3).

As used herein, the term “CL domain”, or “CL domain” can be used interchangeably and refer to the corresponding light chain immunoglobulin constant domain. Unless instructed otherwise, this term may thus encompass a CL domain of the kappa (κ or K) or lambda (a) class of immunoglobulin light chains, including all known subtypes (e.g.)λ1, λ2, λ3, and λ7). In particular, when the CL domain is of the kappa class, it may also be referred herein as a Cκ or CK or Ck domain.

As used herein, the terms “pair C (CH1/CL)”, or “paired C (CH1/CL)” “refers to one constant heavy chain domain 1 and one constant light chain domain (e.g., a kappa (κ or K) or lambda (λ) class of immunoglobulin light chains) bound to one another by covalent or non-covalent bonds, preferably non-covalent bonds; thus forming a heterodimer. Unless specified otherwise, when the constant chain domains forming the pair are not present on a same polypeptide chain, this term may thus encompass all possible combinations. Preferably, the corresponding CH1 and CL domains will thus be selected as complementary to each other, such that they form a stable pair C (CH1/CL).

Advantageously, when the binding protein comprises a plurality of paired C domains, such as one “pair C1 (CH1/CL)” and one “pair C2 (CH1/CL)”, each CH1 and CL domain forming the pairs will be selected so that they are formed between complementary CH1 and CL domains. Examples of complementary CH1 and CL domains have been previously described in the international patent applications WO2006064136 or WO2012089814 or WO2015197593A1.

Unless instructed otherwise, the terms “pair C1 (CH1/CL)” or “pair C2 (CH1/CL)” may refer to distinct constant pair domains (C1 and C2) formed by identical or distinct constant heavy 1 domains (CH1) and identical or distinct constant light chain domains (CL). Preferably, the terms “pair C1 (CH1/CL)” or “pair C2 (CH1/CL)” may refer to distinct constant pair domains (C1 and C2) formed by identical constant heavy 1 domains (CH1) and identical constant light chain domains (CL).

As used herein, the term “Fc region” or “fragment crystallizable region”, or alternatively “Fc portion”, encompasses all or parts of the “Fc domain”, which may thus include all or parts of an immunoglobulin hinge region (which natively bears a first binding site to FcγRs), a CH2 domain (which natively bears a second binding site to FcγRs), and a CH3 domain of an immunoglobulin (e.g. of an IgG, IgA or IgD immunoglobulin), and/or when applicable of a CH4 domain of an immunoglobulin (e.g. for IgM and IgE). Preferably, the Fc region includes all or parts of, at least, a CH2 domain and a CH3 domain, and optionally all or parts of an immunoglobulin hinge region. The term may thus refer to a molecule comprising the sequence of a non-antigen-binding fragment resulting from digestion of an antibody or produced by other means, whether in monomeric or multimeric form, and can contain the hinge region. The original immunoglobulin source of the native Fe is, in particular, of human origin and can be any of the immunoglobulins, although IgG1 are preferred. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 13 depending on class (e.g., IgG, IgA, and IgE) or subclass (e.g., IgG1, IgG2, IgG3, IgGA1, and IgGA2). One example of a native Fc is a disulfide-bonded dimer resulting from papain digestion of an IgG. The term “native Fc” as used herein is generic to the monomeric, dimeric, and multimeric forms. Under that terminology, a “Fc region” may thus comprise or consist of CH2-CH3 (e.g., (CH2-CH3)A or (CH2-CH3)B or a binding pair thereof, and optionally all or part of an immunoglobulin hinge region, comprising a binding site to a human FcγR. Unless specified otherwise, the term “Fc region” may refer to either a native or variant Fc region.

The term “Fc variant” as used herein refers to a molecule or sequence that is modified from a native Fc but still comprises a binding site for the receptor, FcRn (neonatal Fc receptor). Exemplary Fc variants, and their interaction with the receptor, are known in the art. Thus, the term “Fc variant” can comprise a molecule or sequence that is humanized from a non-human native Fc. Furthermore, a native Fc comprises regions that can be removed because they provide structural features or biological activity that are not required for the antibody-like binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3)N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The fragment crystallizable (Fc) regions (e.g., native or variant) according to the present disclosure retain a capacity to bind to a human Fc-7 receptor polypeptide (Fcγ) which generally occurs on native Fc regions through binding of the antibody Fc-hinge region. As a reference, overall structures of IgG1, IgG2, and IgG4 are similar with more than 90% sequence homology, the major differences residing in the hinge region and CH2 domain, which form primary binding sites to FcγRs. The hinge region also functions as a flexible linker between the Fab and Fc portion.

Fc regions having one or more amino acid modifications (e.g., substitutions, deletions, insertions) in one or more portions, which modifications increase the affinity and avidity of the variant Fc region for an FcγR (including activating and inhibitory FcγRs) are further considered as Fc regions. In some embodiments, said one or more amino acid modifications increase the affinity of the Fc region for FcγRIIIA and/or FcγRIIA. In another embodiment, the variant Fc region further specifically binds FcγRIIB with a lower affinity than does the Fc region of the reference parent antibody (e.g., an antibody having the same amino acid sequence as the antibody except for the one or more amino acid modifications in the Fc region). Hence, native and variant Fc regions considered herein generally comprise a domain (i.e., a CH2 domain) capable of binding to human CD16, e.g., a human Fc domain comprising N-linked glycosylation at amino acid residue N297 (according to EU numbering).

As used herein, the term “Fc-competent” thus refers to a binding protein that is capable of binding specifically to a FcγR, in particular of an activating FcγR, in particular to one selected from FcγRI (CD64a), FcγRIIa (CD32a), and FcγRIIIa (CD16a), and more particularly to FcγRIIIa (CD16a).

Alternatively, several modifications are reported to directly affect the binding to FcγRs, including mutation on residues 297 (according to EU numbering), or alternatively on residues 234 and 235 in the lower hinge region (according to the EU numbering system).

As used herein, the term “Fc-silent” refers to a binding protein with a Fc region, wherein the Fc region lacks a binding site to a FcγR (e.g., a Fc region lacking a CH2 domain with said binding site and hinge region with said binding site); in particular FcγRI, FcγRIIa, and FcγRIIIa, and more particularly to FcγRIIIa (CD16a).

As used herein, the term “variable”, as in “variable domain”, refers to certain portions of the relevant binding protein which differ extensively in sequence between and among antibodies and are used in the specific recognition and binding of a particular antibody for its particular target. However, the variability is not evenly distributed throughout the entire variable domains of antibodies. The variability is concentrated in three segments called complementarity determining regions (CDRs; i.e., CDR1, CDR2, and CDR3) also known as hypervariable regions, both in the light chain and the heavy chain variable domains. The more highly conserved portions of variable domains are called the framework (FR) regions or sequences.

As used herein, the term “VH domain”, or “VH domain” can be used interchangeably and refer to the corresponding heavy chain immunoglobulin variable domain.

As used herein, the term “VL domain”, or “VL domain” can be used interchangeably and refer to the corresponding light chain immunoglobulin variable domain.

When the VH or VL domains are associated to a first antigen-binding domain (ABD) or to a second antigen-binding domain, they may also be respectively referred herein as “VH1” and “VL1”, or “VH2” and “VL2”.

The terms “binding pair V (VH/VL)”, “VH/VL pair” or “(VH/VL) pair” or “VL/VH pair” or “(VL/VH) pair” can be used interchangeably. Heavy chain and light chain variable domain can pair in parallel to form the antigen binding domains (ABDs). Each binding pair includes both a VH and a VL region. Unless instructed otherwise, these terms do not specify which immunoglobulin variable regions are VH or VL regions and which ABD will bind specifically the protein expressed on the surface of an immune effector cell or a target cell (e.g., NKp46 and CD123).

As used herein, the term “hypervariable region’ when used herein refers to the amino acid residues of an antibody that are responsible for antigen binding. This term may be substituted by the terms “Complementarity Determining Regions” or “CDRs”.

Thus, as used herein “Complementarity Determining Regions” or “CDRs” refer to amino acid sequences that together define the binding affinity and specificity of the natural Fv region of a native immunoglobulin binding site. The light and heavy chains of an immunoglobulin each have three CDRs, designated CDR-L1, CDR-L2, CDR-L3 and CDR-H1, CDR-H2, CDR-H3, respectively. A conventional antibody antigen-binding domain, therefore, includes six CDRs, comprising the CDR set from each of a heavy and a light chain variable region. Also, as used herein, “Framework Regions” (FRs) refer to amino acid sequences interposed between CDRs, i.e., to those portions of immunoglobulin light and heavy chain variable regions that are relatively conserved among different immunoglobulins in a single species. The light and heavy chains of an immunoglobulin each have four FRs, designated FR-L1, FR-L2, FR-L3, FR-L4, and FR-H1, FR-H2, FR-H3, FR-H4, respectively. Accordingly, the light chain variable domain may thus be designated as (FR-L1)-(CDR-L1)-(FR-L2)-(CDR-L2)-(FR-L3)-(CDR-L3)-(FR-L4) and the heavy chain variable domain may thus be designated as (FR-H1)-(CDR-H1)-(FR-H2)-(CDR-H2)-(FR-H3)-(CDR-H)-(FR4-H3).

The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). The numbering of amino acid residues in this region is performed by the method described in Kabat et al., supra. Accordingly, phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

Optionally, CDRs are as defined by EU, Kabat, Chotia or IMGT numbering. Correspondances between those classifications are known in the Art, by reference to the IMGT®, or international ImMunoGeneTics information system® (CNRS and Montpellier University), and as further detailed in Lefranc (Biomolecules; 2014; 4, 1102-1139) and Dondelinger (Frontiers in Immunology; 2018; 9, 2278). CDRs may also be defined according to the Honegger-Pluckthun (“Honegger”) numbering scheme described in Honnegger and Pluckthun (2001), J. Mol. Biol., vol. 309(3):657-670.

Unless instructed otherwise, the numbering of residues will be considered herein by reference to the EU, Kabat, Chotia, IMGT, or Honegger-Pluckthun numbering convention. In case of conflict regarding the exact position of hypervariable regions within a reference sequence, the Kabat numbering convention will prevail. In case of conflict regarding the exact position of constant regions within a reference sequence, the EU numbering convention will prevail. Further,

As used herein, the term “cytotoxicity” refers to the quality of a compound, such as the multifunctional binding protein according to the present disclosure, to be toxic to tumoral cells. Cytotoxicity may be induced by different mechanisms of action and can thus be divided into cell-mediated cytotoxicity, apoptosis, antibody-dependent cell-mediated cytotoxicity (ADCC), antibody-dependent cellular phagocytosis (ADCP) or complement-dependent cytotoxicity (CDC).

As used herein, the term “antibody-dependent cell-mediated cytotoxicity” or “ADCC” refers to a mechanism of cell-mediated immune defence whereby an effector cell of the immune system actively lyses a target cell, whose membrane-surface antigens have been bound by specific antibodies or the multifunctional binding protein of the present disclosure.

As used herein, the terms “proliferative disorders”, “hyper-proliferative disorders” and/or “cancer” not only refer to solid tumors, such as cancers of the breast, respiratory tract, brain, reproductive organs, digestive tract, urinary tract, eye, liver, skin, head and neck, thyroid, parathyroid and their distant metastases, but also include blood cancers, including tumors of the hematopoietic and lymphoid tissues, such as lymphomas, myelomas, and leukemias. Leukemias include, but are not limited to acute myeloid leukemia, acute lymphoblastic leukemia, chronic lymphocytic leukemia, chronic myelogenous leukemia, and hairy cell leukemia.

As used herein, “Acute myelogenous leukemia (AML)” is a clonal disorder clinically presenting as increased proliferation of heterogeneous and undifferentiated myeloid blasts. Without wishing to be bound by the theory, the leukemic hierarchy is maintained by a small population of LSCs (Leukemic Stem Cells) (AML-LSCs), which have the distinct ability for self-renewal, and are able to differentiate into leukemic progenitors. These progenitors generate the large numbers of leukemic blasts readily detectable in patients at diagnosis and relapse, leading ultimately to mortality. AML-LSC have been commonly reported as quiescent cells, in contrast to rapidly dividing clonogenic progenitors.

Within the context of AML, the term “relapse” may in particular be defined as the reoccurrence of AML after complete remission. In that sense “complete remission” or “CR” may be defined as follows: normal values for neutrophil (>1.0*10⁹/L), haemoglobin level of 10 g/dl and platelet count (>100*10⁹/L) and independence from red cell transfusion; blast cells less than 5%, no clusters or collections of blasts, and absence of Auer rods on bone marrow examination; and normal maturation of blood cells (morphology; myelogramme) and absence of extramedullary leukemia.

As used herein, the term “refractory” means the cancer did not respond to treatment. In the context of AML, most patients achieve a remission (an absence of signs and symptoms) after initial treatment. However, some patients have residual leukemic cells in their marrow even after intensive treatment. Patients who have not achieved complete remission after two cycles of induction chemotherapy are usually diagnosed as having “refractory AML.”

As used herein, “myelodysplastic syndromes” (“MDS”), formerly known as preleukemia, are a collection of hematological conditions that involve ineffective production (or dysplasia) of the myeloid class of blood cells. They represent a spectrum of clonal hematopoietic stem cell disorders characterized by progressive bone marrow failure and increased risk of progression to acute myeloid leukemia (“AML”, also known as “acute myelogenous leukemia”). The International Prognostic Scoring System (“IPSS”) is widely used to identify patients with high-risk features based on the severity of their cytopenias, bone marrow myeloblast percentage, and cytogenetic abnormalities.

As used herein, a “pharmaceutically acceptable carrier” is intended to include any and all carrier (such as any solvents, dispersion media, coatings, antibacterial and antifungal agents, isotonic and absorption delaying agents, and the like) which is compatible with pharmaceutical administration, in particular parenteral administration. The use of such media and agents for pharmaceutically active substances are known. Except insofar as any conventional media or agent is incompatible with the active compound, such media can be used in the compositions of the present disclosure. For example, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In a non-exhaustive manner, pharmaceutically acceptable carriers include, but are not limited to, 0.01-0.1M (e.g., 0.05M) phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringeability exists. It should be stable under the conditions of manufacture and storage and will in an embodiment be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In certain embodiments, isotonic agents are included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

As used herein, and unless instructed otherwise, the term “at least one” may encompass “one or more”, or even “two or more” (or “a plurality”). For instance, it may encompass 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more than 100.

As used herein, and unless instructed otherwise, the term “less than . . . ” may encompass all values from 0 to the corresponding threshold, For instance, it may encompass less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or less than 100, when applicable.

As used herein, the term “cell” may encompass any prokaryotic cell or eukaryotic cell. Cell types which are particularly considered are those suitable for the production and/or engineering of recombinant antibodies, or fragments, or polypeptide chains thereof. In a non-exhaustive manner, such cells may be selected from the group consisting of: bacterial cells, yeast cells, mammalian cells, non-mammalian cells, insect cells, and plant cells.

The terms “host cell,” “host cell line,” and “host cell culture” as used herein, are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein. A host cell is any type of cellular system that can be used to generate binding proteins of the present disclosure. Host cells may thus include cultured cells, e.g., mammalian cultured cells, such as CHO cells, HEK cells, BHK cells, NS0 cells, SP2/0 cells, YO myeloma cells, P3×63 mouse myeloma cells, PER cells, PER.C6 cells or hybridoma cells, bacterial cells, yeast cells, insect cells, and plant cells, to name only a few.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present disclosure. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present disclosure, as well as positive and negative strand forms, and double-stranded forms. Isolated polynucleotides or nucleic acids according to the present disclosure further include such molecules produced synthetically. In addition, a polynucleotide or a nucleic acid may be or may include a regulatory element such as a promoter, ribosome binding site, or a transcription terminator.

The term “vector’ or “expression vector” is intended to mean the vehicle by which a nucleic acid, in particular a DNA or RNA sequence (e.g., a foreign gene), can be introduced into a host cell, so as to transform the host and promote expression (e.g., transcription and translation) of the introduced sequence.

### II. Binding Protein

Provided herein is a binding protein comprising a bispecific NK cell engager (NKCE) with a competent Fc domain that can bind to CD16a (FcγRIIIa) used in methods for treating leukemias and myelodysplastic syndromes. The NKCE of the disclosure functions as a trifunctional molecule that binds NKp46 and CD16a on the surface of the NK cells and CD123 on malignant cells. Co-engagement of a NK cell and a malignant cell by the CD123 NKCE of the present disclosure leads to the formation of an immunological synapse which induces NK-cell activation and degranulation.

In some embodiments, the binding protein is characterized in that it comprises a first antigen binding domain with binding specificity to CD123 and a second antigen binding domain with binding specificity to NKp46. In some embodiments,

In some embodiments, the NKCE is characterized in that it comprises a first antigen binding domain with binding specificity to CD123 and a second antigen binding domain with binding specificity to NKp46, wherein the first antigen binding domain comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the binding protein is characterized in that the first antigen binding domain with binding specificity comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the binding protein is characterized in that it comprises the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, the binding protein is characterized in that it comprises the VH1 comprises an amino acid sequence of SEQ ID NO: 41, and wherein the VL1 comprises an amino acid sequence of SEQ ID NO: 43; or the VH1 comprises an amino acid sequence of SEQ ID NO: 42, and wherein the VL1 comprises an amino acid sequence of SEQ ID NO: 44.

In some embodiments, the binding protein comprises a second antigen binding domain with binding specificity to NKp46. In some embodiments, the second antigen binding domain comprises CDRs defined by Kabat numbering. In some embodiments, the second antigen binding domain comprises CDRs defined by IMGT numbering. In some embodiments, the second antigen binding domain comprises CDRs defined by Chothia numbering. In some embodiments, the second antigen binding domain comprises CDRs defined by Honegger numbering. In some embodiments, the second antigen binding domain comprises CDRs as defined in Table 1.

TABLE 1
Example Anti-NKp46 CDRs

| | Kabat | IMGT | Chothia | Honegger |
|---|---|---|---|---|
| CDRL1 | RASQDI | QDISNY | SQDISNY | ASQDI |
| | SNYLN | (SEQ ID | (SEQ ID | SNY |
| | (SEQ ID | NO: | NO: | (SEQ ID |
| | NO: | 99) | 105) | NO: |
| | 27) | | | 111) |
| CDRL2 | YTSRLHS | YTS | YTS | YTSRLH |
| | (SEQ ID | (SEQ ID | (SEQ ID | SGVPSR |
| | NO: | NO: | NO: | (SEQ ID |
| | 28) | 100) | 106) | NO: |
| | | | | 112) |
| CDRL3 | QQGNTRPWT | QQGNT | GNTRPW | GNTRPW |
| | (SEQ ID | RPWTF | (SEQ ID | (SEQ ID |
| | NO: | (SEQ ID | NO: | NO: |
| | 29) | NO: | 107) | 107) |
| | | 101) | | |
| | | or | | |
| | | QQGNT | | |
| | | RPWT | | |
| | | (SEQ ID | | |
| | | NO: | | |
| | | 116) | | |
| CDRH1 | DYVIN | GYTFSDYV | GYTFSDY | ASGYTF |
| | (SEQ ID | (SEQ ID | (SEQ ID | SDYV |
| | NO: | NO: | NO: | (SEQ ID |
| | 13) | 102) | 108) | NO: |
| | | | | 113) |
| CDRH2 | EIYPGS | IYPGSGTN | PGSG | IYPGSG |
| | GTNYYN | (SEQ ID | (SEQ ID | TNYYNE |
| | EKFKA | NO: | NO: | KFKAK |
| | (SEQ ID | 103) | 109) | (SEQ ID |
| | NO: | | | NO: |
| | 14) | | | 114) |
| CDRH3 | RGRYG | ARRGRY | GRYGL | RGRYGL |
| | LYAMDY | GLYAMDY | YAMD | YAMD |
| | (SEQ ID | (SEQ ID | (SEQ ID | (SEQ ID |
| | NO: | NO: | NO: | NO: |
| | 15) | 104) | 110) | 115) |

In some embodiments, the binding protein is characterized in that the second antigen binding domain with binding specificity to NKp46 comprises:
a. a second heavy chain variable domain (VH2) comprising a CDR-H1, H2, and H3 corresponding to the amino acid sequences of:
   i. SEQ ID NO: 13, 14, and 15, respectively;
   ii. SEQ ID NO: 16, 17, and 18, respectively;
   iii. SEQ ID NO: 19, 20, and 21, respectively;
   iv. SEQ ID NO: 22, 23, and 24, respectively; or
   v. SEQ ID NO: 16, 25, and 26, respectively; and
b. a second light chain variable domain (VL2) comprising a CDR-L1, L2, and L3 corresponding to the amino acid sequences of:
   i. SEQ ID NO: 27, 28, and 29, respectively;
   ii. SEQ ID NO: 30, 31, and 32, respectively;
   iii. SEQ ID NO: 33, 34, and 35, respectively;
   iv. SEQ ID NO: 36, 37, and 38, respectively; or
   v. SEQ ID NO: 39, 31, and 40, respectively.

In some embodiments, the binding protein is characterized in that:
a. the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; or
b. the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54
c. the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55;
d. the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56;
e. the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57;
f. the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58;
g. the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or
h. the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

In some embodiments, the binding protein is characterized in that:
a. the VH2 comprises an amino acid sequence of SEQ ID NO: 45, and wherein the VL1 comprises an amino acid sequence of SEQ ID NO: 53; or
b. the VH2 comprises an amino acid sequence of SEQ ID NO: 46, and wherein the VL2 comprises an amino acid sequence of SEQ ID NO: 54
c. the VH2 comprises an amino acid sequence of SEQ ID NO: 47, and wherein the VL2 comprises an amino acid sequence of SEQ ID NO: 55;
d. the VH2 comprises an amino acid sequence of SEQ ID NO: 48, and wherein the VL2 comprises an amino acid sequence of SEQ ID NO: 56;
e. the VH2 comprises an amino acid sequence of SEQ ID NO: 49, and wherein the VL2 comprises an amino acid sequence of SEQ ID NO: 57;
f. the VH2 comprises an amino acid sequence of SEQ ID NO: 50, and wherein the VL2 comprises an amino acid sequence of SEQ ID NO: 58;
g. the VH2 comprises an amino acid sequence of SEQ ID NO: 51, and wherein the VL2 comprises an amino acid sequence of SEQ ID NO: 59; or
h. the VH2 comprises an amino acid sequence of SEQ ID NO: 52, and wherein the VL2 comprises an amino acid sequence of SEQ ID NO: 60.

In some embodiments, the binding protein is characterized in that the binding protein comprises three polypeptide chains (I), (II) and (III) that form two ABDs, as defined below:

V_{1A}-C_{1A}-Hinge₁-(C_{H}2-C_{H}3)_{A} (I)

V_{1B}-C_{1B}-Hinge₂-(C_{H}2-C_{H}3)_{B}-L₁-V_{2A}-C_{2A}-Hinge₃ (II)

V_{2B}-C_{2B} (III)

wherein:
V_{1A} and V_{1B} form a binding pair V₁(V_{H1}/V_{L1});
V_{2A} and V_{2B} form a binding pair V₂(V_{H2}/V_{L2});
C_{1A} and C_{1B} form a pair C₁(C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
   Hinge₁, Hinge₂ and Hinge₃ are identical or different and correspond to all or part of an immunoglobulin hinge region;
   (C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
   L₁ is an amino acid linker.

In some embodiments, the binding protein is characterized in that it comprises a C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO:75;
Hinge₃ corresponds to the amino acid sequence of SEQ ID NO: 77;
L₁ corresponds to the amino acid sequence of SEQ ID NO: 76.

In some embodiments, the binding protein is characterized in that the residue N297 of the Fc region or variant thereof according to EU numbering comprises a N-linked glycosylation.

In some embodiments, the binding protein is characterized in that the all or part of the Fc region or variant thereof binds to a human CD16A (FcγRIII) polypeptide.

In some embodiments, the binding protein is characterized in that at least two polypeptide chains are linked by at least one disulfide bridge.

In some embodiments, the binding protein is characterized in that the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

In some embodiments, the binding protein is characterized in that V_{1A} is V_{L1} and V_{1B} is V_{H1}.

In some embodiments, the binding protein is characterized in that V_{2A} is V_{H2} and V_{2B} is V_{L2}.

In some embodiments, the binding protein is characterized in that:
(a) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(b) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(d) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38;
(e) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;
(f) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(g) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(h) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(i) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or
(j) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, the binding protein is characterized in that:
(a) V_{H1} and V_{L1} corresponds to the amino acid sequences of SEQ ID NO: 41 and 43 respectively or corresponds to the amino acid sequences of SEQ ID NO: 42 and 44 respectively;
   and/or
(b) V_{H2} and V_{L2} corresponds to
   the amino acid sequences of SEQ ID NO: 45 and 53 respectively;
   the amino acid sequences of SEQ ID NO: 46 and 54 respectively;
   the amino acid sequences of SEQ ID NO: 47 and 55 respectively;
   the amino acid sequences of SEQ ID NO: 48 and 56 respectively;
   the amino acid sequences of SEQ ID NO: 49 and 57 respectively;
   the amino acid sequences of SEQ ID NO: 50 and 58 respectively;
   the amino acid sequences of SEQ ID NO: 51 and 59 respectively; or
   the amino acid sequences of SEQ ID NO: 52 and 60 respectively.

In some embodiments, the binding protein is characterized in that:
(a) V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53;
(b) V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54;
(c) V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55;
(d) V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56;
(e) V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57;
(f) V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58;
(g) V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59;
(h) V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60;
(i) V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53;
(j) V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54;
(k) V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55;
(l) V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56;
(m) V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57;
(n) V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58.
(o) V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59;
(p) V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60.

In some embodiments, the binding protein is characterized in that:
polypeptide (I) consists of an amino acid sequence of SEQ ID NO: 64;
polypeptide (II) consists of an amino acid sequence of SEQ ID NO: 65; and
polypeptide (III) consists of an amino acid sequence of SEQ ID NO: 66.

### III. Methods of Use/Treatment

Provided herein are methods of treating or preventing a hematological disease or disorder in a subject in need thereof comprising administering to the subject a therapeutically effective amount of the K-NK cells of the disclosure and a therapeutically effective amount of the CD123 NKCE of the present disclosure.

Provided herein is a method of treating or preventing a leukemia or a myelodysplastic syndrome in a subject in need thereof, the method comprising administering to the subject a binding protein comprising a first antigen binding domain with binding specificity to CD123 and a second antigen binding domain with binding specificity to NKp46, in combination with a K-NK cell of the present disclosure.

A therapeutically effective dose of the K-NK cells and/or the NK cell engager disclosed herein may be the dose or amount sufficient to induce a “therapeutic response” in a subject, which as an improvement in at least one measure of a hematological disease or disorder (e.g., ALL, B-ALL, or HR-MDS). In some embodiments, the therapeutically effective dose of the NK cell engager is expressed micrograms (μg) per kg of the patient's body weight (μg/kg) or as a flat dose. In some embodiments, the NK cell engager is administered as a serial dosing regimen. In some embodiments, the therapeutically effective dose of the K-NK cell is expressed as cells/administration, cells/kg body weight, or effector cell (E) to target cell (T) ratio (E:T).

In some embodiments, the therapeutically effective dose of the NK cell is between about 1×10⁴ cells/kg and about 1×10⁹ cells/kg. In some embodiments, the therapeutically effective dose of the NK is between about 1×10⁴ cells/kg and about 1×10⁸ cells/kg.

In some embodiments, the therapeutically effective dose of the NK cell engager is between about 1 μg/kg and about 6000 μg/kg. In some embodiments, the therapeutically effective dose is between about 1 μg/kg and about 10 μg/kg. In some embodiments, the therapeutically effective dose is between about 10 and about 100 μg/kg. In some embodiments, the therapeutically effective dose is between about 100 μg/kg and about 150 μg/kg. In some embodiments, the therapeutically effective dose is between about 150 μg/kg and about 200 μg/kg. In some embodiments, the therapeutically effective dose is between about 200 μg/kg and about 250 μg/kg. In some embodiments, the therapeutically effective dose is between about 250 μg/kg and about 300 μg/kg. In some embodiments, the therapeutically effective dose is between about 300 μg/kg and about 350 μg/kg. In some embodiments, the therapeutically effective dose is between about 350 μg/kg and about 400 μg/kg. In some embodiments, the therapeutically effective dose is between about 400 μg/kg and about 450 μg/kg. In some embodiments, the therapeutically effective dose is between about 450 μg/kg and about 500 μg/kg. In some embodiments, the therapeutically effective dose is between about 500 μg/kg and about 550 μg/kg. In some embodiments, the therapeutically effective dose is between about 550 μg/kg and about 600 μg/kg. In some embodiments, the therapeutically effective dose is between about 600 μg/kg and about 650 μg/kg. In some embodiments, the therapeutically effective dose is between about 650 μg/kg and about 700 μg/kg. In some embodiments, the therapeutically effective dose is between about 700 μg/kg and about 750 μg/kg. In some embodiments, the therapeutically effective dose is between about 750 μg/kg and about 800 μg/kg. In some embodiments, the therapeutically effective dose is between about 800 μg/kg and about 850 μg/kg. In some embodiments, the therapeutically effective dose is between about 850 μg/kg and about 900 μg/kg. In some embodiments, the therapeutically effective dose is between about 900 μg/kg and about 950 μg/kg. In some embodiments, the therapeutically effective dose is between about 950 μg/kg and about 1000 μg/kg. In some embodiments, the therapeutically effective dose is between about 1000 μg/kg and about 1300 μg/kg. In some embodiments, the therapeutically effective dose is between about 1300 μg/kg and about 1500 μg/kg. In some embodiments, the therapeutically effective dose is between about 1500 μg/kg and about 2000 μg/kg. In some embodiments, the therapeutically effective dose is between about 2000 μg/kg and about 2500 μg/kg. In some embodiments, the therapeutically effective dose is between about 2500 μg/kg and about 3000 μg/kg. In some embodiments, the therapeutically effective dose is between about 3000 μg/kg and about 4000 μg/kg. In some embodiments, the therapeutically effective dose is between about 4000 μg/kg and about 4500 μg/kg. In some embodiments, the therapeutically effective dose is between about 4500 μg/kg and about 5000 μg/kg. In some embodiments, the therapeutically effective dose is between about 5000 μg/kg and about 6000 μg/kg.

In some embodiments, the therapeutically effective dose is about 3 μg/kg. In some embodiments, the therapeutically effective dose is about 10 μg/kg. In some embodiments, the therapeutically effective dose is about 13 μg/kg. In some embodiments, the therapeutically effective dose is about 15 μg/kg. In some embodiments, the therapeutically effective dose is about 20 μg/kg. In some embodiments, the therapeutically effective dose is about 30 μg/kg. In some embodiments, the therapeutically effective dose is about 40 μg/kg. In some embodiments, the therapeutically effective dose is about 45 μg/kg. In some embodiments, the therapeutically effective dose is about 50 μg/kg. In some embodiments, the therapeutically effective dose is about 60 μg/kg. In some embodiments, the therapeutically effective dose is about 100 μg/kg. In some embodiments, the therapeutically effective dose is about 100 μg/kg. In some embodiments, the therapeutically effective dose is about 130 μg/kg. In some embodiments, the therapeutically effective dose is about 150 μg/kg. In some embodiments, the therapeutically effective dose is about 200 μg/kg. In some embodiments, the therapeutically effective dose is about 300 μg/kg. In some embodiments, the therapeutically effective dose is about 400 μg/kg. In some embodiments, the therapeutically effective dose is about 450 μg/kg. In some embodiments, the therapeutically effective dose is about 600 μg/kg. In some embodiments, the therapeutically effective dose is about 1000 μg/kg. In some embodiments, the therapeutically effective dose is about 1300 μg/kg. In some embodiments, the therapeutically effective dose is about 1500 μg/kg. In some embodiments, the therapeutically effective dose is about 2000 μg/kg. In some embodiments, the therapeutically effective dose is about 3000 μg/kg. In some embodiments, the therapeutically effective dose is about 4000 μg/kg. In some embodiments, the therapeutically effective dose is about 4500 μg/kg. In some embodiments, the therapeutically effective dose is about 6000 μg/kg.

In certain embodiments, the present disclosure provides kits and methods for the treatment of diseases and disorders, e.g., hematological diseases or disorders in a mammalian subject in need of such treatment. In some embodiments, the hematological disease or disorder is acute myeloid leukemia (AML). In some embodiments, the AML is relapsed or refractory.

The binding protein of the current disclosure are useful in a number of different applications. For example, in one embodiment, the subject binding proteins are useful for reducing or eliminating cells bearing an epitope recognized by a binding domain of the binding protein. In another embodiment, the subject binding proteins are effective in reducing the concentration of or eliminating soluble antigen in the circulation. In another embodiment, the subject binding proteins are effective as NK-cell engagers (NKCEs).

In another embodiment, the CD123 NKCEs are useful for the treatment of diseases or disorders associated with aberrant immune cells, e.g., myeloid cells, B cells. In some embodiments, the aberrant immune cells, e.g., myeloid cells or B cells, express CD123.

In some embodiments, the CD123 NKCEs of the present disclosure can be particularly useful in the treatment of a disease or disorder within the category of leukemias or myelodysplasias. In some embodiments, the leukemia is AML.

### Acute Myeloid Leukemia (AML)

Newly diagnosed de novo AML patients capable of undergoing intensive induction therapy typically receive a cytosine arabinoside and anthracycline based induction therapy, followed by consolidation chemotherapy with cytarabine or other agents. Patients with AML are eligible to receive a liposomal formulation of daunorubicin/cytarabine. As the understanding of the molecular drivers of AML have improved, newer molecularly targeted therapies have emerged. Elderly patients (>75 years) or patients that have comorbidities that preclude aggressive chemotherapy induction are treated with less aggressive therapies that include venetoclax/hypomethylating agent combinations which provide better outcomes than prior single agent regimens.

Allogeneic stem cell transplantation is offered to patients with high-risk disease in first or subsequent remissions, provided that they have adequate organ function and a suitable source of stem cells. Although about 15% of patients >60 years of age and 40% of patients <60 years of age who receive intensive induction chemotherapy and/or allogeneic stem cell transplantation for AML are cured; AML patients with relapsed or refractory disease (r/r) following initial therapy exhibit a poor prognosis. Despite the development of several new agents for r/r AML, it is usually incurable. A short duration of remission (e.g., <6 months), adverse genetic factors, prior allogeneic transplantation, older age, and general health status are factors associated with worse survival outcomes in the setting of r/r AML, in part because these factors limit eligibility for further intensive therapy.

The relative 5-year survival rate from 2011 to 2017 for patients with AML was 29.5%. In 2022, 20,050 new cases of AML are estimated in the United States, with a median age at diagnosis of 68 years. Recent clinical trial data indicate a 3-year overall survival rate of only 50% to 60% in adults who have achieved a complete remission. Despite advances in understanding the pathophysiology of AML and recognizing its molecular heterogeneity, developing viable therapeutics for patients with AML is challenging.

### IV. Pharmaceutical Compositions and Administration Thereof

Methods of preparing and administering the K-NK cells and the CD123 NKCE of the present disclosure to a subject are well known to or are readily determined by those skilled in the art.

In some embodiments, the K-NK cells may be administered at a different time or in a different pharmaceutical composition than the NKCE. In some embodiments, the K-NK cells may be administered prior to the NKCE. In some embodiments, the K-NK cells may be administered after the NKCE. In some embodiments, the K-NK cells may be administered at the same time as the NKCE.

In some embodiments, the pharmaceutical composition may comprise an unstimulated NK cell or a stimulated NK cell, or a population of modified immune cells. In some embodiments, the pharmaceutical composition may comprise the NK cells after stimulation with the PM21 particles. In some embodiments, the pharmaceutical composition may comprise the NK cells and PM21 particles.

The route of administration of the K-NK cells and/or the NKCE of the present disclosure may be oral, parenteral, by inhalation, or topical. The term parenteral as used herein includes intravenous, intraarterial, intraperitoneal, intramuscular, subcutaneous, rectal or vaginal administration. While all these forms of administration are clearly contemplated as being within the scope of the present disclosure, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. In some embodiments, the NKCE can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M, e.g., 0.05 M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will typically be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

In many cases, isotonic agents will be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a K-NK cell by itself, an NKCE by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit. Such articles of manufacture will typically have labels or package inserts indicating that the associated compositions are useful for treating a subject suffering from or predisposed to autoimmune or neoplastic disorders.

Effective doses of the compositions of the present disclosure, for the treatment of the above-described conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

The K-NK cells and/or the CD123 NKCE proteins of the present disclosure can be administered on multiple occasions. Intervals between single dosages can be weekly, monthly or yearly. Intervals can also be irregular as indicated by measuring blood levels of Fc domain variant or antigen in the patient. In some methods, dosage is adjusted to achieve a plasma modified binding polypeptide concentration of about 1-1000 g/ml and in some methods about 25-300 g/ml. Alternatively, Fc domain variants can be administered as a sustained release formulation, in which case less frequent administration is required. For antibodies, dosage and frequency vary depending on the half-life of the antibody in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present polypeptides or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from about 0.001 to about 25 mg/kg per dose, especially about 0.003 to about 6.0 mg/kg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of antibody per dose, with dosages of from about 5 to 25 mg being more commonly used for radioimmunoconjugates and higher doses for cytotoxin-drug modified antibodies) at relatively short intervals is sometimes required until progression of the disease is reduced or terminated, or until the patient shows partial or complete amelioration of disease symptoms. Thereafter, the patient can be administered a prophylactic regime.

As previously discussed, the K-NK cell and NKCE combination of the present disclosure, antibodies, therapeutic polypeptides, or NKCE fusion polypeptides thereof, may be administered in a pharmaceutically effective amount for the in vivo treatment of mammalian disorders. In this regard, it will be appreciated that the disclosed K-NK cells and NKCEs will be formulated to facilitate administration and promote stability of the active agent.

A pharmaceutical composition in accordance with the present disclosure can comprise a pharmaceutically acceptable, non-toxic, sterile carrier such as physiological saline, nontoxic buffers, preservatives and the like. For the purposes of the instant application, a pharmaceutically effective amount of the CD123 NKCE, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of plasma cells, the polypeptide can interact with selected antigens on immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the K-NK cells and CD123 NKCEs of the disclosure may be administered to a human or other animal in accordance with the aforementioned methods of treatment in an amount sufficient to produce a therapeutic or prophylactic effect. The K-NK cell sand CD123 NKCEs of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

In some embodiments, disclosed herein are kits for increasing the number of K-NK cells comprising one or more cytokines (for example, IL-12, IL-15, and/or IL-18) and one or more particles comprising an NK cell effector agent. In one aspect, the particle is a PM21 particle. For example, the disclosed kits can comprise IL-12 and PM21 particles; IL-15 and PM21 particles; or IL-18 and PM21 particles. In some embodiments, the kits can be used with NK cells obtained from a donor source including NK cells obtained from an unselected population of peripheral blood mononuclear cells (PBMCs). In some embodiments, the donor source for the NK cells being used can also be the recipient of the NK cells (i.e., autologous). In some embodiments, the NK cells are from an allogeneic donor source.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### Sequence Listing

In the protein sequences notation used herein, the left-hand direction is the amino terminal direction (the “N terminus” or “N-term”) and the right-hand direction is the carboxyl-terminal direction (the “C terminus” or “C-term”), in accordance with standard usage and convention.

| SEQ | Name | Sequence |
|---|---|---|
| 1 | V_{H1} CDR-H1 | GYSFTDYYMK |
| | Anti-CD123 | |
| | CD123-1 | |
| 2 | V_{H1} CDR-H2 | DIIPSSGATF |
| | Anti-CD123 | |
| | CD123-1 | |
| 3 | V_{H1} CDR-H3 | SHLLRASWFAY |
| | Anti-CD123 | |
| | CD123-1 | |
| 4 | Anti-CD123 | GFTFSHYN |
| | CD123-2 | |
| 5 | V_{H1} CDR-H2 | ITYDDHST |
| | Anti-CD123 | |
| | CD123-2 | |
| 6 | V_{H1} CDR-H3 | ARLVNYAFAY |
| | Anti-CD123 | |
| | CD123-2 | |
| 7 | V_{L1} CDR-L1 | ESSQSLLSSGNQKNYLT |
| | Anti-CD123 | |
| | CD123-1 | |
| 8 | V_{L1} CDR-L2 | WASTRES |
| | Anti-CD123 | |
| | CD123-1 | |
| 9 | V_{L1} CDR-L3 | QNDYSYPYT |
| | Anti-CD123 | |
| | CD123-1 | |
| 10 | V_{L1} CDR-L1 | QTVGNN |
| | Anti-CD123 | |
| | CD123-2 | |
| 11 | V_{L1} CDR-L2 | YAS |
| | Anti-CD123 | |
| | CD123-2 | |
| 12 | V_{L1} CDR-L3 | QRMYNSPT |
| | Anti-CD123 | |
| | CD123-2 | |
| 13 | V_{H2} CDR-H1 | DYVIN |
| | Anti-NKp46 | |
| | NKp46-1 | |
| 14 | V_{H2} CDR-H2 | EIYPGSGTNYYNEKFKA |
| | Anti-NKp46 | |
| | 3D9 and NKp46-1 | |
| 15 | V_{H2} CDR-H3 | RGRYGLYAMDY |
| | Anti-NKp46 | |
| | 3D9 and NKp46-1 | |
| 16 | V_{H2} CDR-H1 | SDYAWN |
| | Anti-NKp46 | |
| | NKp46-2 | |
| | 13G4 | |
| 17 | V_{H2} CDR-H2 | YITYSGSTSYNPSLES |
| | Anti-NKp46 | |
| | NKp46-2 | |
| 18 | V_{H2} CDR-H3 | GGYYGSSWGVFAY |
| | Anti-NKp46 | |
| | NKp46-2 | |
| 19 | V_{H2} CDR-H1 | EYTMH |
| | Anti-NKp46 | |
| | NKp46-3 | |
| 20 | V_{H2} CDR-H2 | GISPNIGGTSYNQKFKG |
| | Anti-NKp46 | |
| | NKp46-3 | |
| 21 | V_{H2} CDR-H3 | RGGSFDY |
| | Anti-NKp46 | |
| | NKp46-3 | |
| 22 | V_{H2} CDR-H1 | SFTMH |
| | Anti-NKp46 | |
| | NKp46-4 | |
| 23 | V_{H2} CDR-H2 | YINPSSGYTEYNQKFKD |
| | Anti-NKp46 | |
| | NKp46-4 | |
| 24 | V_{H2} CDR-H3 | GSSRGFDY |
| | Anti-NKp46 | |
| | NKp46-4 | |
| 25 | V_{H2} CDR-H2 | YITYSGSTNYNPSLKS |
| | Anti-NKp46 | |
| | 13G4 | |
| 26 | V_{H2} CDR-H3 | CWDYALYAMDC |
| | Anti-NKp46 | |
| | 13G4 | |
| 27 | V_{L2} CDR-H1 | RASQDISNYLN |
| | Anti-NKp46 | |
| | 3D9 and NKp46-1 | |
| 28 | V_{L2} CDR-H2 | YTSRLHS |
| | Anti-NKp46 | |
| | 3D9 and NKp46-1 | |
| 29 | V_{L2} CDR-H3 | QQGNTRPWT |
| | Anti-NKp46 | |
| | 3D9 and NKp46-1 | |
| 30 | V_{L2} CDR-H1 | RVSENIYSYLA |
| | Anti-NKp46 | |
| | NKp46-2 | |
| 31 | V_{L2} CDR-H2 | NAKTLAE |
| | Anti-NKp46 | |
| | NKp46-2 | |
| | 13G4 | |
| 32 | V_{L2} CDR-H3 | QHHYGTPWT |
| | Anti-NKp46 | |
| | NKp46-2 | |
| 33 | V_{L2} CDR-H1 | RASQSISDYLH |
| | Anti-NKp46 | |
| | NKp46-3 | |
| 34 | V_{L2} CDR-H2 | YASQSIS |
| | Anti-NKp46 | |
| | NKp46-3 | |
| 35 | V_{L2} CDR-H3 | QNGHSFPLT |
| | Anti-NKp46 | |
| | NKp46-3 | |
| 36 | V_{L2} CDR-H1 | RASENIYSNLA |
| | Anti-NKp46 | |
| | NKp46-4 | |
| 37 | V_{L2} CDR-H2 | AATNLAD |
| | Anti-NKp46 | |
| | NKp46-4 | |
| 38 | V_{L2} CDR-H3 | QHFWGTPRT |
| | Anti-NKp46 | |
| | NKp46-4 | |
| 39 | V_{L2} CDR-H2 | RTSENIYSYLA |
| | Anti-NKp46 | |
| | 13G4 | |
| 40 | V_{L2} CDR-H3 | QHHYDTPLT |
| | Anti-NKp46 | |
| | 13G4 | |
| 41 | VH-full length- | EVQLVQSGAEVKKPGESLKISCKGS |
| | anti-CD123 | GYSFTDYYMKWARQMPGKGLEWMGD |
| | | IIPSSGATFYNQKFKGQVTISADKS |
| | | ISTTYLQWSSLKASDTAMYYCARSH |
| | | LLRASWFAYWGQGTMVTVSS |
| 42 | VH-full length- | EVQLVESGGGLVQPGRSLKLSCAAS |
| | anti-CD123 | GFTFSHYNMAWVRQAPKKGLEWVAT |
| | | ITYDDHSTYYRDSVKGRFTISRDTA |
| | | KSTLYLQMDSLRSEDTATYYCARLV |
| | | NYAFAYWGQGTLVTVSS |
| 43 | VL-full length- | DIVMTQSPDSLAVSLGERATINCES |
| | anti-CD123 | SQSLLSSGNQKNYLTWYQQKPGQPP |
| | | KPLIYWASTRESGVPDRFSGSGSGT |
| | | DFTLTISSLQAEDVAVYYCQNDYSY |
| | | PYTFGQGTKLEIK |
| 44 | VL-full length- | NIVMTQSPKSMSISVGDRVTMNCKA |
| | anti-CD123 | SQTVGNNIAWYQQKPGLSPQLLIDY |
| | | ASNRYTGVPNRFTGGGYGTDFILTI |
| | | NSVQAEDAAFYYCQRMYNSPTFGGG |
| | | TKLELK |
| 45 | VH-full length- | QVQLVQSGAEVKKPGSSVKVSCKAS |
| | anti-NKp46 | GYTFSDYVINWVRQAPGQGLEWMGE |
| | | IYPGSGTNYYNEKFKAKATITADKS |
| | | TSTAYMELSSLRSEDTAVYYCARRG |
| | | RYGLYAMDYWGQGTTVTVSS |
| 46 | VH-full length- | QVQLQQSGPELVKPGASVKMSCKAS |
| | anti-NKp46 | GYTFTDYVINWGKQRSGQGLEWIGE |
| | NKp46-1 | IYPGSGTNYYNEKFKAKATLTADKS |
| | | SNIAYMQLSSLTSEDSAVYFCARRG |
| | | RYGLYAMDYWGQGTSVTVSS |
| 47 | VH-full length- | EVQLQESGPGLVKPSQSLSLTCTVT |
| | anti-NKp46 | GYSITSDYAWNWIRQFPGNKLEWMG |
| | NKp46-2 | YITYSGSTSYNPSLESRISITRDTS |
| | | TNQFFLQLNSVTTEDTATYYCARGG |
| | | YYGSSWGVFAYWGQGTLVTVSA |
| 48 | VH-full length- | EVQLQQSGPELVKPGASVKISCKTS |
| | anti-NKp46 | GYTFTEYTMHWVKQSHGKSLEWIGG |
| | NKp46-3 | ISPNIGGTSYNQKFKGKATLTVDKS |
| | | SSTAYMELRSLTSEDSAVYYCARRG |
| | | GSFDYWGQGTTLTVSS |
| 49 | VH-full length- | QVQLQQSAVELARPGASVKMSCKAS |
| | anti-NKp46 | GYTFTSFTMHWVKQRPGQGLEWIGY |
| | NKp46-4 | INPSSGYTEYNQKFKDKTTLTADKS |
| | | SSTAYMQLDSLTSDDSAVYYCVRGS |
| | | SRGFDYWGQGTLVTVSA |
| 50 | VH-full length- | QVQLQESGPGLVKPSQTLSLTCTVS |
| | anti-NKp46 | GYSISSDYAWNWIRQPPGKGLEWIG |
| | 10B8 | YITYSGSTSYNPSLESRVTISRDTS |
| | | KNQFSLKLSSVTAADTAVYYCARGG |
| | | YYGSSWGVFAYWGQGTLVTVSS |
| 51 | VH-full length- | QVQLVQSGAEVKKPGASVKVSCKAS |
| | anti-NKp46 | GYTFTSFTMHWVRQAPGQGLEWIGY |
| | 12E12 | INPSSGYTEYNQKFKDRVTITADKS |
| | | TSTAYMELSSLRSEDTAVYYCVRGS |
| | | SRGFDYWGQGTLVTVSS |
| 52 | VH-full length- | QVQLQESGPGLVKPSQTLSLTCTVS |
| | anti-NKp46 | GYSISSDYAWNWIRQPPGKGLEWIG |
| | 13G4 | YITYSGSTNYNPSLKSRVTISRDTS |
| | | KNQFSLKLSSVTAADTAVYYCARCW |
| | | DYALYAMDCWGQGTTVTVSS |
| 53 | VL-full length- | DIQMTQSPSSLSASVGDRVTITCRA |
| | anti-NKp46 | SQDISNYLNWYQQKPGKAPKLLIYY |
| | 3D9 | TSRLHSGVPSRFSGSGSGTDFTFTI |
| | | SSLQPEDIATYFCQQGNTRPWTFGG |
| | | GTKVEIK |
| 54 | VL-full length- | DIQMTQTTSSLSASLGDRVTISCRA |
| | anti-NKp46 | SQDISNYLNWYQQKPDGTVKLLIYY |
| | NKp46-1 | TSRLHSGVPSRFSGSGSGTDYSLTI |
| | | NNLEQEDIATYFCQQGNTRPWTFGG |
| | | GTKLEIK |
| 55 | VL-full length- | DIQMTQSPASLSASVGETVTITCRV |
| | anti-NKp46 | SENIYSYLAWYQQKQGKSPQLLVYN |
| | NKp46-2 | AKTLAEGVPSRFSGSGSGTQFSLKI |
| | | NSLQPEDFGSYYCQHHYGTPWTFGG |
| | | GTKLEIK |
| 56 | VL-full length- | DIVMTQSPATLSVTPGDRVSLSCRA |
| | anti-NKp46 | SQSISDYLHWYQQKSHESPRLLIKY |
| | NKp46-3 | ASQSISGIPSRFSGSGSGSDFTLSI |
| | | NSVEPEDVGVYYCQNGHSFPLTFGA |
| | | GTKLELK |
| 57 | VL-full length- | DIQMIQSPASLSVSVGETVTITCRA |
| | anti-NKp46 | SENIYSNLAWFQQKQGKSPQLLVYA |
| | NKp46-4 | ATNLADGVPSRFSGSGSGTQYSLKI |
| | | NSLQSEDFGIYYCQHFWGTPRTFGG |
| | | GTKLEIK |
| 58 | VL-full length- | DIQMTQSPSSLSASVGDRVTITCRV |
| | anti-NKp46 | SENIYSYLAWYQQKPGKAPKLLVYN |
| | 10B8 | AKTLAEGVPSRFSGSGSGTDFTLTI |
| | | SSLQPEDFATYYCQHHYGTPWTFGG |
| | | GTKVEIK |
| 59 | VL-full length- | DIQMTQSPSSLSASVGDRVTITCRA |
| | anti-NKp46 | SENIYSNLAWFQQKPGKAPKLLVYA |
| | 12E12 | ATNLADGVPSRFSGSGSGTDYTLTI |
| | | SSLQPEDFATYYCQHFWGTPRTFGG |
| | | GTKVEIK |
| 60 | VL-full length- | DIQMTQSPSSLSASVGDRVTITCRT |
| | anti-NKp46 | SENIYSYLAWCQQKPGKAPKLLIYN |
| | 13G4 | AKTLAEGVPSRFSGSGSGTDFTLTI |
| | | SSLQPEDFATYYCQHHYDTPLTFGQ |
| | | GTKLEIK |
| 61 | Polypeptide chain | MSVPTQVLGLLLLWLTDARCDIVMT |
| | (I) | QSPDSLAVSLGERATINCESSQSLL |
| | | SSGNQKNYLTWYQQKPGQPPKPLIY |
| | | WASTRESGVPDRFSGSGSGTDFTLT |
| | | ISSLQAEDVAVYYCQNDYSYPYTFG |
| | | QGTKLEIKRTVAAPSVFIFPPSDEQ |
| | | LKSGTASVVCLLNNFYPREAKVQWK |
| | | VDNALQSGNSQESVTEQDSKDSTYS |
| | | LSSTLTLSKADYEKHKVYACEVTHQ |
| | | GLSSPVTKSFNRGECDKTHTCPPCP |
| | | APELLGGPSVFLFPPKPKDTLMISR |
| | | TPEVTCVVVDVSHEDPEVKFNWYVD |
| | | GVEVHNAKTKPREEQYNSTYRVVSV |
| | | LTVLHQDWLNGKEYKCKVSNKALPA |
| | | PIEKTISKAKGQPREPQVYTLPPSR |
| | | EEMTKNQVSLTCLVKGFYPSDIAVE |
| | | WESNGQPENNYKTTPPVLDSDGSFF |
| | | LYSKLTVDKSRWQQGNVFSCSVMHE |
| | | ALHNHYTQKSLSLSPGK |
| 62 | Polypeptide chain | MEWSWVFLFFLSVTTGVHSEVQLVQ |
| | (II) | SGAEVKKPGESLKISCKGSGYSFTD |
| | | YYMKWARQMPGKGLEWMGDIIPSSG |
| | | ATFYNQKFKGQVTISADKSISTTYL |
| | | QWSSLKASDTAMYYCARSHLLRASW |
| | | FAYWGQGTMVTVSSASTKGPSVFPL |
| | | APSSKSTSGGTAALGCLVKDYFPEP |
| | | VTVSWNSGALTSGVHTFPAVLQSSG |
| | | LYSLSSVVTVPSSSLGTQTYICNVN |
| | | HKPSNTKVDKRVEPKSCDKTHTCPP |
| | | CPAPELLGGPSVFLFPPKPKDTLMI |
| | | SRTPEVTCVVVDVSHEDPEVKFNWY |
| | | VDGVEVHNAKTKPREEQYNSTYRVV |
| | | SVLTVLHQDWLNGKEYKCKVSNKAL |
| | | PAPIEKTISKAKGQPREPQVYTLPP |
| | | SREEMTKNQVSLTCLVKGFYPSDIA |
| | | VEWESNGQPENNYKTTPPVLDSDGS |
| | | FFLYSKLTVDKSRWQQGNVFSCSVM |
| | | HEALHNHYTQKSLSLSPGSTGSQVQ |
| | | LVQSGAEVKKPGSSVKVSCKASGYT |
| | | FSDYVINWVRQAPGQGLEWMGEIYP |
| | | GSGTNYYNEKFKAKATITADKSTST |
| | | AYMELSSLRSEDTAVYYCARRGRYG |
| | | LYAMDYWGQGTTVTVSSASTKGPSV |
| | | FPLAPSSKSTSGGTAALGCLVKDYF |
| | | PEPVTVSWNSGALTSGVHTFPAVLQ |
| | | SSGLYSLSSVVTVPSSSLGTQTYIC |
| | | NVNHKPSNTKVDKRVEPKSCDKTHS |
| 63 | Polypeptide chain | MSVPTQVLGLLLLWLTDARCDIQMT |
| | (III) | QSPSSLSASVGDRVTITCRASQDIS |
| | | NYLNWYQQKPGKAPKLLIYYTSRLH |
| | | SGVPSRFSGSGSGTDFTFTISSLQP |
| | | EDIATYFCQQGNTRPWTFGGGTKVE |
| | | IKRTVAAPSVFIFPPSDEQLKSGTA |
| | | SVVCLLNNFYPREAKVQWKVDNALQ |
| | | SGNSQESVTEQDSKDSTYSLSSTLT |
| | | LSKADYEKHKVYACEVTHQGLSSPV |
| | | TKSFNRGEC |
| 64 | F25 polypeptide | DIVMTQSPDSLAVSLGERATINCES |
| | chain (I) | SQSLLSSGNQKNYLTWYQQKPGQPP |
| | Cleaved (without | KPLIYWASTRESGVPDRFSGSGSGT |
| | leader peptide) | DFTLTISSLQAEDVAVYYCQNDYSY |
| | | PYTFGQGTKLEIKRTVAAPSVFIFP |
| | | PSDEQLKSGTASVVCLLNNFYPREA |
| | | KVQWKVDNALQSGNSQESVTEQDSK |
| | | DSTYSLSSTLTLSKADYEKHKVYAC |
| | | EVTHQGLSSPVTKSFNRGECDKTHT |
| | | CPPCPAPELLGGPSVFLFPPKPKDT |
| | | LMISRTPEVTCVVVDVSHEDPEVKF |
| | | NWYVDGVEVHNAKTKPREEQYNSTY |
| | | RVVSVLTVLHQDWLNGKEYKCKVSN |
| | | KALPAPIEKTISKAKGQPREPQVYT |
| | | LPPSREEMTKNQVSLTCLVKGFYPS |
| | | DIAVEWESNGQPENNYKTTPPVLDS |
| | | DGSFFLYSKLTVDKSRWQQGNVFSC |
| | | SVMHEALHNHYTQKSLSLSPGK |
| 65 | F25 polypeptide | EVQLVQSGAEVKKPGESLKISCKGS |
| | chain (II) | GYSFTDYYMKWARQMPGKGLEWMGD |
| | Cleaved (without | IIPSSGATFYNQKFKGQVTISADKS |
| | leader peptide) | ISTTYLQWSSLKASDTAMYYCARSH |
| | | LLRASWFAYWGQGTMVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKRVEPKSCDK |
| | | THTCPPCPAPELLGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPSREEMTKNQVSLTCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPGS |
| | | TGSQVQLVQSGAEVKKPGSSVKVSC |
| | | KASGYTFSDYVINWVRQAPGQGLEW |
| | | MGEIYPGSGTNYYNEKFKAKATITA |
| | | DKSTSTAYMELSSLRSEDTAVYYCA |
| | | RRGRYGLYAMDYWGQGTTVTVSSAS |
| | | TKGPSVFPLAPSSKSTSGGTAALGC |
| | | LVKDYFPEPVTVSWNSGALTSGVHT |
| | | FPAVLQSSGLYSLSSVVTVPSSSLG |
| | | TQTYICNVNHKPSNTKVDKRVEPKS |
| | | CDKTHS |
| 66 | F25 polypeptide | DIQMTQSPSSLSASVGDRVTITCRA |
| | chain (III) | SQDISNYLNWYQQKPGKAPKLLIYY |
| | Cleaved (without | TSRLHSGVPSRFSGSGSGTDFTFTI |
| | leader peptide) | SSLQPEDIATYFCQQGNTRPWTFGG |
| | | GTKVEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| 67 | Cκ | RTVAAPSVFIFPPSDEQLKSGTASV |
| | | VCLLNNFYPREAKVQWKVDNALQSG |
| | | NSQESVTEQDSKDSTYSLSSTLTLS |
| | | KADYEKHKVYACEVTHQGLSSPVTK |
| | | SFNRGEC |
| 68 | C_{H}1 | ASTKGPSVFPLAPSSKSTSGGTAAL |
| | | GCLVKDYFPEPVTVSWNSGALTSGV |
| | | HTFPAVLQSSGLYSLSSVVTVPSSS |
| | | LGTQTYICNVNHKPSNTKVDKRV |
| 69 | (C_{H}2-C_{H}3)A | APELLGGPSVFLFPPKPKDTLMISR |
| | | TPEVTCVVVDVSHEDPEVKFNWYVD |
| | | GVEVHNAKTKPREEQYNSTYRVVSV |
| | | LTVLHQDWLNGKEYKCKVSNKALPA |
| | | PIEKTISKAKGQPREPQVYTLPPSR |
| | | EEMTKNQVSLTCLVKGFYPSDIAVE |
| | | WESNGQPENNYKT |
| | | TPPVLDSDGSFFLYSKLTVDKSRWQ |
| | | QGNVFSCSVMHEALHNHYTQKSLSL |
| | | SPGK |
| 70 | (C_{H}2-C_{H}3)B | APELLGGPSVFLFPPKPKDTLMISR |
| | | TPEVTCVVVDVSHEDPEVKFNWYVD |
| | | GVEVHNAKTKPREEQYNSTYRVVSV |
| | | LTVLHQDWLNGKEYKCKVSNKALPA |
| | | PIEKTISKAKGQPREPQVYTLPPSR |
| | | EEMTKNQVSLTCLVKGFYPSDIAVE |
| | | WESNGQPENNYKTTPPVLDSDGSFF |
| | | LYSKLTVDKSRWQQGNVFSCSVMHE |
| | | ALHNHYTQKSLSLSPG |
| 71 | C_{H}2 | APELLGGPSVFLFPPKPKDTLMISR |
| | | TPEVTCVVVDVSHEDPEVKFNWYVD |
| | | GVEVHNAKTKPREEQYNSTYRVVSV |
| | | LTVLHQDWLNGKEYKCKVSNKALPA |
| | | PIEKTISKAK |
| 72 | C_{H}3 of | GQPREPQVYTLPPSREEMTKNQVSL |
| | (C_{H}2-C_{H}3)_{A} | TCLVKGFYPSDIAVEWESNGQPENN |
| | | YKTTPPVLDSDGSFFLYSKLTVDKS |
| | | RWQQGNVFSCSVMHEALHNHYTQKS |
| | | LSLSPGK |
| 73 | C-term | GQPREPQVYTLPPSREEMTKNQVSL |
| | truncated | TCLVKGFYPSDIAVEWESNGQPENN |
| | C_{H}3 of | YKTTPPVLDSDGSFFLYSKLTVDKS |
| | (C_{H}2-C_{H}3)_{B} | RWQQGNVFSCSVMHEALHNHYTQKS |
| | | LSLSPG |
| 74 | Hinge₁ | DKTHTCPPCP |
| 75 | Hinge₂ | EPKSCDKTHTCPPCP |
| 76 | Linker₁ | STGS |
| 77 | Hinge₃ | EPKSCDKTHS |
| 78 | Hinge_{ALT1} | EPKSCDKTH |
| 79 | Hinge_{ALT2} | EPKSCDKTHT |
| 80 | Human NKp46 | MSSTLPALLCVGLCLSQRISAQQQT |
| | extracellular | LPKPFIWAEPHFMVPKEKQVTICCQ |
| | domain (ECD) | GNYGAVEYQLHFEGSLFAVDRPKPP |
| | | ERINKVKFYIPDMNSRMAGQYSCIY |
| | | RVGELWSEPSNLLDLVVTEMYDTPT |
| | | LSVHPGPEVISGEKVTFYCRLDTAT |
| | | SMFLLLKEGRSSHVQRGYGKVQAEF |
| | | PLGPVTTAHRGTYRCFGSYNNHAWS |
| | | FPSEPVKLLVTGDIENTSLAPEDPT |
| | | FPADTWGTYLLTTETGLQKDHALWD |
| | | HTAQN |
| 81 | Cynomolgus | MSSTLRALLCLGLCLSQRISAPKQT |
| | NKp46-FlagM2 | LPKPIIRAESTYMVPKEKQATLCCQ |
| | extracellular | GSYGAVEYQLHFEGSLFAVERPKPP |
| | domain (ECD) | ERINGVKFHIPDMNSRKAGRYSCIY |
| | | RVGELWSERSDLLDLVVTEMYDTPT |
| | | LSVHPGPEVTSGEKVTFYCRLDTAT |
| | | SMFLLLKEGRSRDVQRSYGKVQAEF |
| | | PMGPVTTAHRGSYRCFGSYNNYAWS |
| | | FPSEPVKLLVTGDIENTSLAPTDPT |
| | | FPDSWDTCLLTRETGLQKDLALWDH |
| | | TAQNDYKDDDDK |
| 82 | Human CD123 | TKEDPNPPITNLRMKAKAQQLTWDL |
| | extracellular | NRNVTDIECVKDADYSMPAVNNSYC |
| | domain (ECD) | QFGAISLCEVTNYTVRVANPPFSTW |
| | | ILFPENSGKPWAGAENLTCWIHDVD |
| | | FLSCSWAVGPGAPADVQYDLYLNVA |
| | | NRRQQYECLHYKTDAQGTRIGCRFD |
| | | DISRLSSGSQSSHILVRGRSAAFGI |
| | | PCTDKFVVFSQIEILTPPNMTAKCN |
| | | KTHSFMHWKMRSHFNRKFRYELQIQ |
| | | KRMQPVITEQVRDRTSFQLLNPGTY |
| | | TVQIRARERVYEFLSAWSTPQRFEC |
| | | DQEEGANTRAWR |
| 83 | Human Fc- | MWQLLLPTALLLLVSAGMRTEDLPK |
| | gamma-receptor | AVVFLEPQWYRVLEKDSVTLKCQGA |
| | 3A (CD16A). | YSPEDNSTQWFHNESLISSQASSYF |
| | | IDAATVDDSGEYRCQTNLSTLSDPV |
| | | QLEVHIGWLLLQAPRWVFKEEDPIH |
| | | LRCHSWKNTALHKVTYLQNGKGRKY |
| | | FHHNSDFYIPKATLKDSGSYFCRGL |
| | | VGSKNVSSETVNITITQGLAVSTIS |
| | | SFFPPGYQVSFCLVMVLLFAVDTGL |
| | | YFSVKTNIRSSTRDWKDHKFKWRKD |
| | | PQDK |
| 84 | Human Fc- | MWQLLLPTALLLLVSAGMRTEDLPK |
| | gamma-receptor | AVVFLEPQWYRVLEKDSVTLKCQGA |
| | 3A (CD16A) | YSPEDNSTQWFHNESLISSQASSYF |
| | (V176F | IDAATVDDSGEYRCQTNLSTLSDPV |
| | polymorphic | QLEVHIGWLLLQAPRWVFKEEDPIH |
| | variant) | LRCHSWKNTALHKVTYLQNGKGRKY |
| | | FHHNSDFYIPKATLKDSGSYFCRGL |
| | | FGSKNVSSETVNITITQGLAVSTIS |
| | | SFFPPGYQVSFCLVMVLLFAVDTGL |
| | | YFSVKTNIRSSTRDWKDHKFKWRKD |
| | | PQDK |
| 85 | NKp46-IC_F25 | EIVLTQSPATLSLSPGERATLSCRA |
| | Fragment 1 | SQSVRSYLAWYQQKPGQAPRLLFSD |
| | | ASNRATGIPARFSGSGSGTDFTLTI |
| | | SSLEPEDFAVYYCQQYRYSPRTFGQ |
| | | GTKVEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGECDKTHTCPPCPA |
| | | PELLGGPSVFLFPPKPKDTLMISRT |
| | | PEVTCVVVDVSHEDPEVKFNWYVDG |
| | | VEVHNAKTKPREEQYNSTYRVVSVL |
| | | TVLHQDWLNGKEYKCKVSNKALPAP |
| | | IEKTISKAKGQPREPQVYTLPPSRE |
| | | EMTKNQVSLTCLVKGFYPSDIAVEW |
| | | ESNGQPENNYKTTPPVLDSDGSFFL |
| | | YSKLTVDKSRWQQGNVFSCSVMHEA |
| | | LHNHYTQKSLSLSPGK |
| 86 | NKp46-IC_F25 | EVQLVESGGGLVQPGRSLRLSCAAS |
| | Fragment 2 | GFTFDNYAMHWVRQAPGKGLEWVSG |
| | | ISRSSGDIDYADSVKGRFTISRDNA |
| | | KNSLYLQMNSLRAEDTALYYCARGG |
| | | VGSFDTWGQGTMVTVSSASTKGPSV |
| | | FPLAPSSKSTSGGTAALGCLVKDYF |
| | | PEPVTVSWNSGALTSGVHTFPAVLQ |
| | | SSGLYSLSSVVTVPSSSLGTQTYIC |
| | | NVNHKPSNTKVDKRVEPKSCDKTHT |
| | | CPPCPAPELLGGPSVFLFPPKPKDT |
| | | LMISRTPEVTCVVVDVSHEDPEVKF |
| | | NWYVDGVEVHNAKTKPREEQYNSTY |
| | | RVVSVLTVLHQDWLNGKEYKCKVSN |
| | | KALPAPIEKTISKAKGQPREPQVYT |
| | | LPPSREEMTKNQVSLTCLVKGFYPS |
| | | DIAVEWESNGQPENNYKTTPPVLDS |
| | | DGSFFLYSKLTVDKSRWQQGNVFSC |
| | | SVMHEALHNHYTQKSLSLSPGSTGS |
| | | QVQLVQSGAEVKKPGSSVKVSCKAS |
| | | GYTFSDYVINWVRQAPGQGLEWMGE |
| | | IYPGSGTNYYNEKFKAKATITADKS |
| | | TSTAYMELSSLRSEDTAVYYCARRG |
| | | RYGLYAMDYWGQGTTVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKRVEPKSCDK |
| | | THS |
| 87 | NKp46-IC_F25 | DIQMTQSPSSLSASVGDRVTITCRA |
| | Fragment 3 | SQDISNYLNWYQQKPGKAPKLLIYY |
| | | TSRLHSGVPSRFSGSGSGTDFTFTI |
| | | SSLQPEDIATYFCQQGNTRPWTFGG |
| | | GTKVEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| 88 | moNKp46- | DIVMTQSPDSLAVSLGERATINCES |
| | huCD123_F25 | SQSLLSSGNQKNYLTWYQQKPGQPP |
| | Fragment 1 | KPLIYWASTRESGVPDRFSGSGSGT |
| | | DFTLTISSLQAEDVAVYYCQNDYSY |
| | | PYTFGQGTKLEIKRTVAAPSVFIFP |
| | | PSDEQLKSGTASVVCLLNNFYPREA |
| | | KVQWKVDNALQSGNSQESVTEQDSK |
| | | DSTYSLSSTLTLSKADYEKHKVYAC |
| | | EVTHQGLSSPVTKSFNRGECDKTHT |
| | | CPPCPAPELLGGPSVFLFPPKPKDT |
| | | LMISRTPEVTCVVVDVSHEDPEVKF |
| | | NWYVDGVEVHNAKTKPREEQYNSTY |
| | | RVVSVLTVLHQDWLNGKEYKCKVSN |
| | | KALPAPIEKTISKAKGQPREPQVYT |
| | | LPPSREEMTKNQVSLTCLVKGFYPS |
| | | DIAVEWESNGQPENNYKTTPPVLDS |
| | | DGSFFLYSKLTVDKSRWQQGNVFSC |
| | | SVMHEALHNHYTQKSLSLSPGK |
| 89 | moNKp46- | EVQLVQSGAEVKKPGESLKISCKGS |
| | huCD123_F25 | GYSFTDYYMKWARQMPGKGLEWMGD |
| | Fragment 2 | IIPSSGATFYNQKFKGQVTISADKS |
| | | ISTTYLQWSSLKASDTAMYYCARSH |
| | | LLRASWFAYWGQGTMVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKRVEPKSCDK |
| | | THTCPPCPAPELLGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPSREEMTKNQVSLTCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPGS |
| | | TGSEVQLVESGGGLVKPGGSLKLSC |
| | | AASGFTFSDYGMHWVRQAPEKGLEW |
| | | VAYISSGSSTIYYADTVKGRFTISR |
| | | DNAKNTLFLQMTSLRSEDTAMYYCA |
| | | RGTTIFNYFEYWGQGTSVTVSSAST |
| | | KGPSVFPLAPSSKSTSGGTAALGCL |
| | | VKDYFPEPVTVSWNSGALTSGVHTF |
| | | PAVLQSSGLYSLSSVVTVPSSSLGT |
| | | QTYICNVNHKPSNTKVDKRVEPKSC |
| | | DKTHS |
| 90 | moNKp46- | DIVMSQSPSSLAVSVGEKVTMSCKS |
| | huCD123_F25 | SQSLLYSSNQKNYLAWYQQKPGQSP |
| | Fragment 3 | KLLIYWASTRESGVPDRFTGSGSGT |
| | | DFTLTISSVKAEDLAVYYCQQYYEI |
| | | PPTFGAGTKLELKRTVAAPSVFIFP |
| | | PSDEQLKSGTASVVCLLNNFYPREA |
| | | KVQWKVDNALQSGNSQESVTEQDSK |
| | | DSTYSLSSTLTLSKADYEKHKVYAC |
| | | EVTHQGLSSPVTKSFNRGEC |
| 91 | NKp46- | DIVMTQSPDSLAVSLGERATINCES |
| | CD123_F5 | SQSLLSSGNQKNYLTWYQQKPGQPP |
| | Fragment 1 | KPLIYWASTRESGVPDRFSGSGSGT |
| | | DFTLTISSLQAEDVAVYYCQNDYSY |
| | | PYTFGQGTKLEIKRTVAAPSVFIFP |
| | | PSDEQLKSGTASVVCLLNNFYPREA |
| | | KVQWKVDNALQSGNSQESVTEQDSK |
| | | DSTYSLSSTLTLSKADYEKHKVYAC |
| | | EVTHQGLSSPVTKSFNRGECDKTHT |
| | | CPPCPAPELLGGPSVFLFPPKPKDT |
| | | LMISRTPEVTCVVVDVSHEDPEVKF |
| | | NWYVDGVEVHNAKTKPREEQYNSTY |
| | | RVVSVLTVLHQDWLNGKEYKCKVSN |
| | | KALPAPIEKTISKAKGQPREPQVYT |
| | | LPPSREEMTKNQVSLTCLVKGFYPS |
| | | DIAVEWESNGQPENNYKTTPPVLDS |
| | | DGSFFLYSKLTVDKSRWQQGNVFSC |
| | | SVMHEALHNHYTQKSLSLSPGK |
| 92 | NKp46- | EVQLVQSGAEVKKPGESLKISCKGS |
| | CD123_F5 | GYSFTDYYMKWARQMPGKGLEWMGD |
| | Fragment 2 | IIPSSGATFYNQKFKGQVTISADKS |
| | | ISTTYLQWSSLKASDTAMYYCARSH |
| | | LLRASWFAYWGQGTMVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKRVEPKSCDK |
| | | THTCPPCPAPELLGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPSREEMTKNQVSLTCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPGS |
| | | TGSQVQLVQSGAEVKKPGSSVKVSC |
| | | KASGYTFSDYVINWVRQAPGQGLEW |
| | | MGEIYPGSGTNYYNEKFKAKATITA |
| | | DKSTSTAYMELSSLRSEDTAVYYCA |
| | | RRGRYGLYAMDYWGQGTTVTVSSRT |
| | | VAAPSVFIFPPSDEQLKSGTASVVC |
| | | LLNNFYPREAKVQWKVDNALQSGNS |
| | | QESVTEQDSKDSTYSLSSTLTLSKA |
| | | DYEKHKVYACEVTHQGLSSPVTKSF |
| | | NRGEC |
| 93 | NKp46- | DIQMTQSPSSLSASVGDRVTITCRA |
| | CD123_F5 | SQDISNYLNWYQQKPGKAPKLLIYY |
| | Fragment 3 | TSRLHSGVPSRFSGSGSGTDFTFTI |
| | | SSLQPEDIATYFCQQGNTRPWTFGG |
| | | GTKVEIKASTKGPSVFPLAPSSKST |
| | | SGGTAALGCLVKDYFPEPVTVSWNS |
| | | GALTSGVHTFPAVLQSSGLYSLSSV |
| | | VTVPSSSLGTQTYICNVNHKPSNTK |
| | | VDKRVEPKSCDKTHS |
| 94 | IC-CD123_F5 | DIVMTQSPDSLAVSLGERATINCES |
| | Fragment 1 | SQSLLSSGNQKNYLTWYQQKPGQPP |
| | | KPLIYWASTRESGVPDRFSGSGSGT |
| | | DFTLTISSLQAEDVAVYYCQNDYSY |
| | | PYTFGQGTKLEIKRTVAAPSVFIFP |
| | | PSDEQLKSGTASVVCLLNNFYPREA |
| | | KVQWKVDNALQSGNSQESVTEQDSK |
| | | DSTYSLSSTLTLSKADYEKHKVYAC |
| | | EVTHQGLSSPVTKSFNRGECDKTHT |
| | | CPPCPAPELLGGPSVFLFPPKPKDT |
| | | LMISRTPEVTCVVVDVSHEDPEVKF |
| | | NWYVDGVEVHNAKTKPREEQYNSTY |
| | | RVVSVLTVLHQDWLNGKEYKCKVSN |
| | | KALPAPIEKTISKAKGQPREPQVYT |
| | | LPPSREEMTKNQVSLTCLVKGFYPS |
| | | DIAVEWESNGQPENNYKTTPPVLDS |
| | | DGSFFLYSKLTVDKSRWQQGNVFSC |
| | | SVMHEALHNHYTQKSLSLSPGK |
| 95 | IC-CD123_F5 | EVQLVQSGAEVKKPGESLKISCKGS |
| | Fragment 2 | GYSFTDYYMKWARQMPGKGLEWMGD |
| | | IIPSSGATFYNQKFKGQVTISADKS |
| | | ISTTYLQWSSLKASDTAMYYCARSH |
| | | LLRASWFAYWGQGTMVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKRVEPKSCDK |
| | | THTCPPCPAPELLGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPSREEMTKNQVSLTCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPGS |
| | | TGSEVQLVQSGAEVKKSGESLKISC |
| | | KGSGYSFTSYWIGWVRQMPGKGLEW |
| | | MGIFYPGDSSTRYSPSFQGQVTISA |
| | | DKSVNTAYLQWSSLKASDTAMYYCA |
| | | RRRNWGNAFDIWGQGTMVTVSSRTV |
| | | AAPSVFIFPPSDEQLKSGTASVVCL |
| | | LNNFYPREAKVQWKVDNALQSGNSQ |
| | | ESVTEQDSKDSTYSLSSTLTLSKAD |
| | | YEKHKVYACEVTHQGLSSPVTKSFN |
| | | RGEC |
| 96 | IC-CD123_F5 | EIVLTQSPGTLSLSPGERATLSCRA |
| | Fragment 3 | SQSVSSSYLAWYQQKPGQAPRLLIY |
| | | GASSRATGIPDRESGSGSGTDFTLT |
| | | ISRLEPEDFAVYYCQQYGSSTWTFG |
| | | QGTKVEIKASTKGPSVFPLAPSSKS |
| | | TSGGTAALGCLVKDYFPEPVTVSWN |
| | | SGALTSGVHTFPAVLQSSGLYSLSS |
| | | VVTVPSSSLGTQTYICNVNHKPSNT |
| | | KVDKRVEPKSCDKTHS |
| 97 | IC-hIGg1-ADCC- | EIVLTQSPATLSLSPGERATLSCRA |
| | enh | SQSVRSYLAWYQQKPGQAPRLLFSD |
| | Light Chain | ASNRATGIPARFSGSGSGTDFTLTI |
| | | SSLEPEDFAVYYCQQYRYSPRTFGQ |
| | | GTKVEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| 98 | IC-hIGg1-ADCC- | EVQLVESGGGLVQPGRSLRLSCAAS |
| | enh | GFTFDNYAMHWVRQAPGKGLEWVSG |
| | Heavy Chain | ISRSSGDIDYADSVKGRFTISRDNA |
| | | KNSLYLQMNSLRAEDTALYYCARGG |
| | | VGSFDTWGQGTMVTVSSASTKGPSV |
| | | FPLAPSSKSTSGGTAALGCLVKDYF |
| | | PEPVTVSWNSGALTSGVHTFPAVLQ |
| | | SSGLYSLSSVVTVPSSSLGTQTYIC |
| | | NVNHKPSNTKVDKRVEPKSCDKTHT |
| | | CPPCPAPELLGGPDVFLFPPKPKDT |
| | | LMISRTPEVTCVVVDVSHEDPEVKF |
| | | NWYVDGVEVHNAKTKPREEQYNSTY |
| | | RVVSVLTVLHQDWLNGKEYKCKVSN |
| | | KALPAPEEKTISKAKGQPREPQVYT |
| | | LPPSREEMTKNQVSLTCLVKGFYPS |
| | | DIAVEWESNGQPENNYKTTPPVLDS |
| | | DGSFFLYSKLTVDKSRWQQGNVFSC |
| | | SVMHEALHNHYTQKSLSLSPGK |

### EXAMPLES

### Example 1: In Vitro Cytotoxicity Assay of CD123-NKCE and K-NK Cells

### PM21 Particle Preparation

PM particles were prepared from K562-mb21-41BBL cells as previously described (Oyer et al., Generation of highly cytotoxic natural killer cells for treatment of acute myelogenous leukemia using a feeder-free, particle-based approach. Biol Blood Marrow Transplant. 2015. 21: 632-9). Cells were grown in RPMI-1640 media supplemented with 5% fetal bovine serum. Cells were harvested by centrifugation (1000×g, 10 min), washed with Dulbecco's phosphate buffered saline containing 2 mM ethylenediaminetetraacetic acid. Cells were re-suspended in lysis buffer containing 50 mM HEPES, pH 7.4, 150 mM NaCl, 2 mM MgCl₂ and AEBSF, aprotinin, leupeptin and pepstatin A. Cells were disrupted by nitrogen cavitation at 300 psi for 30 min at 4° C. Cell lysate was centrifuged (1000×g, 10 min) and the supernatant was then centrifuged (100,000×g) to pellet the crude cell membranes. The crude membranes were further purified by sucrose gradient centrifugation, and the fraction that corresponds to closed plasma membrane vesicles was collected. All procedures were performed using aseptic techniques and sterility of the product was tested in culture. PM particle preparations were quantified by protein concentration by BCA assay and specified as micrograms of membrane protein/mL. Presence of IL-21 and 41BBL on PM particles was confirmed by enzyme-linked immunosorbent assay and Western blot.

### Cells

The human AML cell line THP-1 that expressed GFP were used in the study. THP-1 cells were used as they express high levels of CD123. THP-1 cells were transfected with the Incucyte® Nuclight Green Lentivirus in order to express Green Fluorescent Protein (GFP). The THP-1 GFP cells were cultured in RPMI 1640 supplemented with 10% fetal bovine serum (FBS), 1% L-Glutamine, 0.05 mM 2-mercaptoethanol, and 1 μg/mL of puromycin to maintain the selectio pressure.

The K-NK cells were isolated from healthy donors and expanded using the PM21 particles as previously described before being frozen and kept at −150° C. (Oyer et al., Generation of highly cytotoxic natural killer cells for treatment of acute myelogenous leukemia using a feeder-free, particle-based approach. Biol Blood Marrow Transplant. 2015. 21: 632-9). Briefly, PBMCs were seeded at 0.1×10⁶ NK cells/mL in stem cell growth medium supplemented with 10% fetal bovine serum, 2 mM Glutamax, 100 U/mL IL02 and 200 μg/mL PM21 particles. Media with supplements was replaced routinely every 2-3 days after day 5.

To perform the in vitro cytotoxicity assay, K-NK cells were thawed and resuspended in 1 mL of complete medium (RPMI 1640 supplemented with 10% FBS and 1% L-glutamine). Cells were transferred in 15 mL conical tube containing 4 mL of complete medium and counted. K-NK cells were then seeded at 1×10⁶ cells/mL in complete medium supplemented with interleukin-2 (50 U/mL) and incubated at 30° C. with 5% CO₂ overnight before being used.

### K-NK Cell Characterization

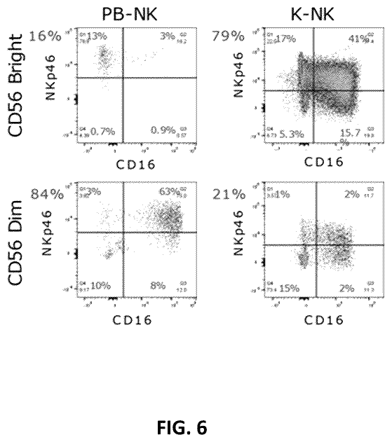

To investigate if K-NK cells (e.g., cells activated with PM21 particles) would be useful to combine with the CD123 NKCE of the present disclosure, cells were stained for expression of certain markers, e.g., NKp46 and CD16 and analyzed using flow cytometry. NKp46 and CD16 were expressed higher in the CD56^{bright} population, compared to NK cells that were not activated with PM21 particles. K-NK cells demonstrated higher expression of NKp46 and CD16 compared to the NK cells isolated from the peripheral blood without PM21 particle activation. Further, the K-NK cells were mainly CD56^{bright}, while the NK cells that were not activated with PM21 particles were mostly CD56^{dim} (FIG. 6). This higher co-expression of NKp46 and CD16 is expected to lead to more optimal engagement of the CD123 NKCE with the K-NK cells.

### Assay

Cytotoxicity was evaluated using the Incucyte live-cells analysis system which allows to quantify the number of live fluorescent target cells over time. First, the CD123-NKCE or its isotype control (molecule specific for human CD123 and Clostridium difficile toxin B with a Fc-silent domain) were distributed at 0.1, 1, 10, 100, 1000 and 10000 ng/mL in the appropriate wells of a flat-bottom 96-well plate coated with poly-D-lysine.

The THP-1 GFP target cells (T) and effector K-NK cells (E) were successively added in each well to obtain E:T ratios of 1:1 (20000 E and 20000 T tumor cells) and 3:1 (60000 E and 20000 T tumor cells). In some wells, only THP-1 GFP cells were seeded and used to monitor the basal rate of tumor cell proliferation. Each condition was performed at least in duplicate.

The growth/proliferation of tumor cells was monitored by cell imaging up to 72 hours using the Incucyte® S3 with standard scans every 4 hours. The Incucyte S3 software was used to analyze images. Tumor cell growth/proliferation was calculated by counting the number of cells at each time point and normalized to the number of cells at time zero. Growth/proliferation was expressed as a percentage.

### Data Analysis

Data generated were exported on Excel files and analyzed. The measured parameter was the count of THP-1 cells normalized by time zero expressed in percentage.

The doses 0.1 ng/mL and 10000 ng/mL were not considered for the statistical analysis but are still presented in the descriptive tables. Due to a high cytotoxicity response at the E:T ratio of 3:1, two donor samples were excluded from the analysis.

### Statistical Analysis

The statistical analysis corresponded to the following objectives for both E:T ratios:
Comparison of SAR445419 (K-NK cells) in presence of Isotype control at each dose to THP-1 alone to evaluate the cytotoxic activity of K-NK cells
Comparison of SAR445419 (K-NK cells) in combination with SAR443579 to SAR445419 in combination with Isotype control at each dose for evaluation of compound cytotoxicity
   For each replicate, the Area Under the Curve (AUC) was computed from 0 h to 72 h according to the following formula:

$$AUC = {\sum }_{h = 0}^{72} 0.5 \times ( h - {h}_{- 1} ) \times ( {Count}_{h} + {Count}_{{h}_{- 1}} )$$

### Where:

h is hour increasing in steps of 4
h₋₁ is the previous measured timepoint
Count is the number of normalized THP−1 cells measured

For each E:T ratio, areas under the curves were summarized by compound using descriptive statistics (Mean, Standard deviation (SD), Median, First (Q1) and Third (Q3) Quartile). Statistical analysis was performed independently for each E:T ratio.

Experimental validation—For each E:T ratio, a mixed model was performed on AUC from SAR445419 combined with Isotype control and THP1-alone with:
Fixed factor Group (concatenation of compound and dose information)
Random factor Donor and Donor*Compound
   A Dunnett's adjustment on pairwise comparisons to THP-1 alone was performed by dose to correct p-values for multiplicity. The statistical analyses were performed using SAS 9.4 for Windows 10. R version 4.1.2 was used for graphical representations. A probability less than 5% (p<0.05) was considered as significant.

### Results

The in vitro cytotoxic activity of the K-NK cells from healthy donors in combination with a CD123 NKCE against THP-1 GFP cells was assessed overtime (up to 72 hours) by Incucyte. The CD123 NKCE or its isotype control were tested at different concentrations (0.1, 1, 10, 100, 1000 and 10000 ng/mL) and at two different E:T ratios (1:1 and 3:1). Doses of 0.1 ng/mL and 10000 ng/mL were not considered for the statistical analysis. All data related to the AUC for each compound at each dose by E:T ratio are presented in Table 2.

TABLE 2
Descriptive table of AUC for each compound at each dose by E:T ratio

| E:T ratio | Compound | Dose (ng/mL) | | N | Mean | SD | Median | [Q1; Q3] |
|---|---|---|---|---|---|---|---|---|
| 0 | THP-1 alone | 0 | | 6 | 11484 | 709 | 11338 | [11138; 12136] |
| 1:1 | SAR445419 + IC | 1e4 | ng/mL | 6 | 9732 | 3206 | 10422 | [6510; 12787] |
| | | 1000 | ng/mL | 6 | 9551 | 3258 | 9831 | [6483; 12827] |
| | | 100 | ng/mL | 5 | 9248 | 3664 | 10288 | [5633; 12238] |
| | | 10 | ng/mL | 6 | 9488 | 2932 | 10548 | [6022; 11955] |
| | | 1 | ng/mL | 6 | 9300 | 3185 | 10236 | [5409; 12265] |
| | | 0.1 | ng/mL | 6 | 9154 | 2827 | 10274 | [5855; 10494] |
| | SAR445419 + SAR443579 | 1e4 | ng/mL | 6 | 8638 | 3846 | 9485 | [4360; 12279] |
| | | 1000 | ng/mL | 6 | 7141 | 3947 | 7920 | [2496; 10452] |
| | | 100 | ng/mL | 6 | 6820 | 3877 | 7472 | [2307; 10298] |
| | | 10 | ng/mL | 6 | 7290 | 3799 | 8446 | [2962; 10651] |
| | | 1 | ng/mL | 6 | 8530 | 3937 | 9538 | [3807; 11763] |
| | | 0.1 | ng/mL | 6 | 8808 | 3005 | 10343 | [5003; 10930] |
| 3:1 | SAR445419 + IC | 1e4 | ng/mL | 4 | 8871 | 1747 | 8625 | [7440; 10303] |
| | | 1000 | ng/mL | 4 | 7972 | 1724 | 7841 | [6908; 9036] |
| | | 100 | ng/mL | 4 | 8263 | 1646 | 8093 | [7005; 9520] |
| | | 10 | ng/mL | 4 | 8118 | 1737 | 7926 | [6863; 9373] |
| | | 1 | ng/mL | 4 | 8008 | 1459 | 7758 | [6827; 9189] |
| | | 0.1 | ng/mL | 4 | 8251 | 1508 | 7748 | [7167; 9335] |
| | SAR445419 + SAR443579 | 1e4 | ng/mL | 4 | 7086 | 2458 | 5906 | [5788; 8385] |
| | | 1000 | ng/mL | 4 | 3829 | 1786 | 3504 | [2602; 5057] |
| | | 100 | ng/mL | 4 | 3992 | 1356 | 3828 | [3144; 4841] |
| | | 10 | ng/mL | 4 | 4784 | 1613 | 4813 | [3631; 5937] |
| | | 1 | ng/mL | 4 | 6828 | 1693 | 6541 | [5629; 8028] |
| | | 0.1 | ng/mL | 4 | 8308 | 1638 | 8166 | [7038; 9577] |

Abbreviations:
SD: Standard deviation;
Q1: first quartile;
Q3: third quartile

### E:T Ratio of 1:1

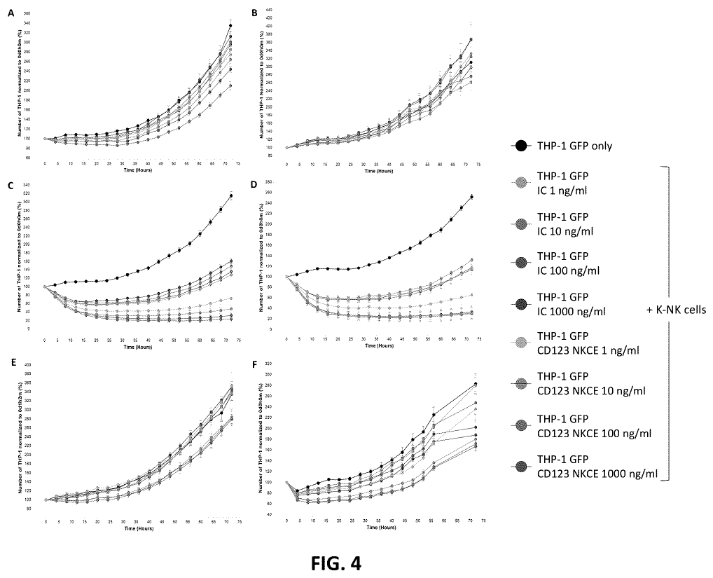

The K-NK cells in the presence of each dose of the isotype control did not induce cytotoxicity of THP-1 cells (no significant difference compared to THP-1 alone; Table 3 and Table 4). However, K-NK cells in the presence of each dose of the CD123 NKCE significantly increased cytotoxicity of THP-1 cells compared to the K-NK cells in the presence of the equivalent dose of the isotype control, and the most potent cytotoxic effect was observed with the CD123 NKCE at 100 ng/mL and 1000 ng/mL (Table 5, Table 6 and FIG. 4).

TABLE 3
K-NK cytotoxicity at E:T ratio of 1:1.

| Effect | p-value |
|---|---|
| Group | 0.2610 |

P-value is obtained with from a mixed model on raw data
For single effects, p-values significant at level 5% are presented in bold.

TABLE 4
Comparison of Isotype Control (IC) at ET ratio of 1:1 to THP-1 alone at each dose.

| | Estimated | Adj. 95% Confidence | | Adjusted |
|---|---|---|---|---|
| Compound versus THP-1 alone | difference | Interval | P-value | p-value |
| SAR445419 + IC 1000 ng/mL | −1814 | [−4916; 1287] | 0.1617 | 0.2097 |
| SAR445419 + IC 100 ng/mL | −2120 | [−5220; 980] | 0.1130 | 0.1486 |
| SAR445419 + IC 10 ng/mL | −2054 | [−5156; 1047] | 0.1219 | 0.1599 |
| SAR445419 + IC 1 ng/mL | −2188 | [−5290; 914] | 0.1041 | 0.1373 |

Adjusted p-values are obtained from a mixed model with a Dunnett'test for multiplicity correction
Adjusted p-values significant at level 5% are presented in bold.

TABLE 5
Compound effect at E:T ratio of 1:1.

| Effect | p-value |
|---|---|
| Compound | <.0001 |
| Dose | 0.0003 |
| Compound *Dose | <.0001 |

P-value is obtained with from a mixed model on raw data
For single effects, p-values significant at level 5% are presented in bold.
For interaction effects, p-values significant at level 10% are presented in bold.

TABLE 6
Comparison between the isotype control and CD123
NKCE + K-NK cells at a E:T ratio of 1:1.

| Dose | Estimated | 95% Confidence | | Adjusted |
|---|---|---|---|---|
| (ng/mL) | difference | Interval | P-value | p-value |
| 1000 | 2497 | [1962; 3031] | <.0001 | <.0001 |
| 100 | 2470 | [1919; 3021] | <.0001 | <.0001 |
| 10 | 2109 | [1574; 2643] | <.0001 | <.0001 |
| 1 | 769 | [248; 1291] | 0.0044 | 0.0044 |

Adjusted p-values are obtained from a mixed model with a Bonferroni-Holm's correction for multiplicity correction
Adjusted p-values significant at level 5% are presented in bold.

### E:T Ratio of 3:1

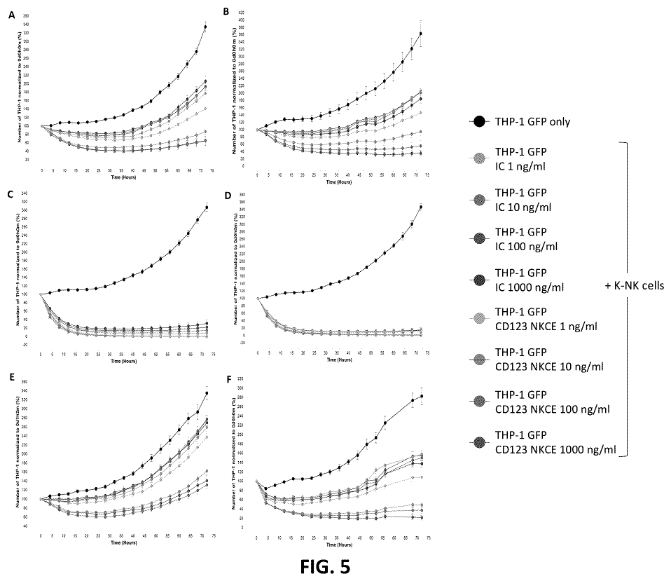

The K-NK cells in the presence of each dose of the isotype control induced strong cytotoxic activity against THP-1 tumor cells (Table 7, Table 8 and FIG. 5). Two K-NK cell donor samples showed very high cytotoxic response at this E:T ratio even in absence of the CD123 NKCE, and were therefore excluded from the analysis. For each dose of the CD123 NKCE, the cytotoxic activity of the K-NK cells was significantly different to that obtained with the equivalent dose of isotype control. A dose-effect of the CD123 NKCE was observed, with the most potent cytotoxic effect induced by the CD123 NKCE at both 100 ng/mL and 1000 ng/mL.

TABLE 7
K-NK cytotoxicity at E:T ratio of 3:1

| Effect | p-value |
|---|---|
| Group | 0.0001 |

P-value is obtained with from a mixed model on raw data
For single effects, p-values significant at level 5% are presented in bold.

TABLE 8
Comparison of K-NK cells and isotype control at
a E:T ratio of 3:1 to THP-1 cells at each dose.

| | Estimated | Adj. 95% Confidence | | Adjusted |
|---|---|---|---|---|
| Compound versus THP-1 alone | difference | Interval | P-value | p-value |
| SAR445419 + IC 1000 ng/mL | −3628 | [−5366; −1890] | 0.0018 | 0.0033 |
| SAR445419 + IC 100 ng/mL | −3337 | [−5075; −1600] | 0.0025 | 0.0046 |
| SAR445419 + IC 10 ng/mL | −3482 | [−5220; −1744] | 0.0021 | 0.0039 |
| SAR445419 + IC 1 ng/mL | −3592 | [−5330; −1854] | 0.0019 | 0.0034 |

Adjusted p-values are obtained from a mixed model with a Dunnett'test for multiplicity correction
Adjusted p-values significant at level 5% are presented in bold.

TABLE 9
Compounds effect at ET ratio of 3:1.

| Effect | p-value |
|---|---|
| Compound | <.0001 |
| Dose | <.0001 |
| Compound*Dose | <.0001 |

P-value is obtained with from a mixed model on raw data
For single effects, p-values significant at level 5% are presented in bold.
For interaction effects, p-values significant at level 10% are presented in bold.

TABLE 10
Comparison between isotype control and CD123
NKCE + K-NK cells at E:T ratio of 3:1.

| Dose | Estimated | 95% Confidence | | Adjusted |
|---|---|---|---|---|
| (ng/mL) | difference | Interval | P-value | p-value |
| 1000 | 4143 | [3709; 4576] | <.0001 | <.0001 |
| 100 | 4270 | [3837; 4704] | <.0001 | <.0001 |
| 10 | 3334 | [2900; 3768] | <.0001 | <.0001 |
| 1 | 1180 | [746; 1613] | <.0001 | <.0001 |

Adjusted p-values are obtained from a mixed model with a Bonferroni-Holm's correction for multiplicity correction
Adjusted p-values significant at level 5% are presented in bold.

1. A composition comprising a Natural killer (NK) cell and a binding protein comprising a first antigen binding domain with binding specificity to CD123 and a second antigen binding domain with binding specificity to NKp46.

2. The composition of claim 1, wherein the NK cell:
is from an autologous donor source,
is from an allogeneic donor source;
is expanded in vitro;
is expanded in vivo;
is isolated from a donor sample prior to being expanded;
is expanded in the presence of other donor sample cells (e.g., PBMCs); or
is expanded by contact with a PM particle.

3-8. (canceled)

9. The composition of claim 2, wherein the PM particle comprises a membrane-bound interleukin 21 (IL-21) molecule and a 4-1BBL molecule (e.g., a PM21 particle); and/or further comprises IL-12 for the expansion of the NK cell.

10. (canceled)

11. The composition according to claim 1, wherein first antigen binding domain with binding specificity to CD123 comprises:
a) a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively,
b) the VH comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43;
c) the VH1 comprises an amino acid sequence that is at least about 90% identical to the amino acid sequence of SEQ ID NO: 42, and wherein the VL comprises and amino acid sequence that is at least about 90% identical to the amino acid sequence of SEQ ID NO: 44;
d) the VH1 comprises the amino acid sequence of SEQ ID NO: 41, and wherein the VL1 comprises the amino acid sequence of SEQ ID NO: 43; and/or
e) the VH1 comprises the amino acid sequence of SEQ ID NO: 42, and wherein the VL1 comprises the amino acid sequence of SEQ ID NO: 44.

12-13. (canceled)

14. The composition according to claim 1, wherein the second antigen binding domain with binding specificity to NKp46 comprises:
a. a second heavy chain variable domain (VH2) comprising a CDR-H1, H2, and H3 corresponding to the amino acid sequences of:
i. SEQ ID NO: 13, 14, and 15, respectively;
ii. SEQ ID NO: 16, 17, and 18, respectively;
iii. SEQ ID NO: 19, 20, and 21, respectively;
iv. SEQ ID NO: 22, 23, and 24, respectively; or
v. SEQ ID NO: 16, 25, and 26, respectively; and
b. a second light chain variable domain (VL2) comprising a CDR-L1, L2, and L3 corresponding to the amino acid sequences of:
i. SEQ ID NO: 27, 28, and 29, respectively;
ii. SEQ ID NO: 30, 31, and 32, respectively;
iii. SEQ ID NO: 33, 34, and 35, respectively;
iv. SEQ ID NO: 36, 37, and 38, respectively; or
v. SEQ ID NO: 39, 31, and 40, respectively.

15. The composition according to claim 1, wherein:
a. the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53;
b. the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54;
c. the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55;
d. the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56;
e. the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57;
f. the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58;
g. the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or
h. the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

16. (canceled)

17. The composition according to claim 1, wherein the binding protein comprises three polypeptide chains (I), (II) and (III) that form two ABDs, as defined below:
V_{1A}-C_{1A}-Hinge₁-(C_{H}2-C_{H}3)_{A} (I)
V_{1B}-C_{1B}-Hinge₂-(C_{H}2-C_{H}3)_{B}-L₁-V_{2A}-C_{2A}-Hinge₃ (II)
V_{2B}-C_{2B} (III)
wherein
V_{1A} and V_{1B} form a binding pair V₁(V_{H1}/V_{L1});
V_{2A} and V_{2B} form a binding pair V₂(V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
L₁ is an amino acid linker; optionally wherein
C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO:75;
Hinge₃ corresponds to the amino acid sequence of SEQ ID NO: 77;
L₁ corresponds to the amino acid sequence of SEQ ID NO: 76.

18. (canceled)

19. The composition according to claim 1, wherein:
residue N297 of the Fc region or variant thereof according to EU numbering comprises a N-linked glycosylation;
the all or part of the Fc region or variant thereof binds to a human CD16A (FcγRIII) polypeptide; and/or
the composition comprises at least two polypeptide chains are liked byy at least one disulfide bridge, optionally wherein the polypeptide chains (I and (II) are linked by at least one disulfide bridge between C_{1A} and Hinge₂ and/or wherein the polypeptide chains (II) and (III) are linked by at least one disulfide bridge between Hinge₃ and C_{2B}.

20-22. (canceled)

23. The composition according to claim 17, wherein V_{1A} is V_{L1} and V_{1B} is V_{H1} and/or V_{2A} is VH2 and V_{2B} is VL2.

24. (canceled)

25. The composition according to claim 1, wherein:
(a) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(b) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(d) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38;
(e) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;
(f) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(g) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(h) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(i) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or
(j) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

26. The composition according to claim 1, wherein:
(a) V_{H1} and V_{L1} corresponds to the amino acid sequences of SEQ ID NO: 41 and 43 respectively or corresponds to the amino acid sequences of SEQ ID NO: 42 and 44 respectively; and/or
(b) V_{H2} and V_{L2} corresponds to
the amino acid sequences of SEQ ID NO: 45 and 53 respectively;
the amino acid sequences of SEQ ID NO: 46 and 54 respectively;
the amino acid sequences of SEQ ID NO: 47 and 55 respectively;
the amino acid sequences of SEQ ID NO: 48 and 56 respectively;
the amino acid sequences of SEQ ID NO: 49 and 57 respectively;
the amino acid sequences of SEQ ID NO: 50 and 58 respectively;
the amino acid sequences of SEQ ID NO: 51 and 59 respectively; or
the amino acid sequences of SEQ ID NO: 52 and 60 respectively; optionally wherein:
(c) V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53;
(d) V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54;
(e) V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55;
(f) V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56;
(g) V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; VL₂ comprises the amino acid sequence of SEQ ID NO: 57;
(h) V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58;
(i) V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59;
(j) V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; VL₂ comprises the amino acid sequence of SEQ ID NO: 60;
(k) V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; VL₂ comprises the amino acid sequence of SEQ ID NO: 53;
(l) V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54;
(m) V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55;
(n) V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56;
(o) V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57;
(p) V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58;
(q) V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59;
(r) V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60; and/or
wherein polypeptide (I) consists of an amino acid sequence of SEQ ID NO: 64, polypeptide (II) consists of an amino acid sequence of SEQ ID NO; 65, and polypeptide (III) consists of an amino acid sequence of SEQ ID NO: 66.

27-28. (canceled)

29. A method of treating or preventing a hematological disease or disorder in a subject in need thereof, the method comprising administering a Natural Killer (NK) cell and a binding protein comprising a first antigen binding domain with binding specificity to CD123 and a second antigen binding domain with binding specificity to NKp46.

30. (canceled)

31. The method of claim 29, wherein the NK cell:
is from an autologous donor source,
is from an allogeneic donor source;
is expanded in vitro;
is expanded in vivo;
is isolated from a donor sample prior to being expanded;
is expanded in the presence of other donor sample cells (e.g., PBMCs); and/or
is expanded by contact with a PM particle, optionally wherein the PM particle comprises a membrane-bound interleukin 21 (IL-21) molecule and a 4-1BBL molecule (e.g., a PM21 particle) and/or further comprises IL-12 for the expansion of the NK cell.

32-39. (canceled)

40. The method according to claim 29, wherein:
the NK cell and the binding protein are administered to the subject intravenously, subcutaneously, intraperitoneally, or intramuscularly, optionally wherein the NK cell and the binding protein are administered to the subject intravenously;
the NK cell and the binding protein are administered at different times, optionally wherein:
the NK cell is administered prior to administration of the binding protein; or
the NK cell is administered after administration of the binding protein; and/or
the NK cell and the binding protein are administered at the same time.

41-45. (canceled)

46. The method of claim 29, wherein the first antigen binding domain with binding specificity to CD123 comprises:
a. a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively;
b. the VH comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43;
c. the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44;
d. the VH1 comprises an amino acid sequence of SEQ ID NO: 41, and wherein the VL1 comprises an amino acid sequence of SEQ ID NO: 43; and/or
e. the VH1 comprises an amino acid sequence of SEQ ID NO: 42, and wherein the VL1 comprises an amino acid sequence of SEQ ID NO: 44.

47-48. (canceled)

49. The method according to claim 29, wherein the second antigen binding domain with binding specificity to NKp46 comprises:
a. a second heavy chain variable domain (VH2) comprising a CDR-H1, H2, and H3 corresponding to the amino acid sequences of:
i. SEQ ID NO: 13, 14, and 15, respectively;
ii. SEQ ID NO: 16, 17, and 18, respectively;
iii. SEQ ID NO: 19, 20, and 21, respectively;
iv. SEQ ID NO: 22, 23, and 24, respectively; or
v. SEQ ID NO: 16, 25, and 26, respectively; and
b. a second light chain variable domain (VL2) comprising a CDR-L1, L2, and L3 corresponding to the amino acid sequences of:
i. SEQ ID NO: 27, 28, and 29, respectively;
ii. SEQ ID NO: 30, 31, and 32, respectively;
iii. SEQ ID NO: 33, 34, and 35, respectively;
iv. SEQ ID NO: 36, 37, and 38, respectively; or
v. SEQ ID NO: 39, 31, and 40, respectively.

50. The method according to claim 29, wherein:
a. the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53;
b. the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54;
c. the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55;
d. the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56;
e. the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57;
f. the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58;
g. the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or
h. the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

51. (canceled)

52. The method according to claim 29, wherein the binding protein comprises three polypeptide chains (I), (II) and (III) that form two ABDs, as defined below:
V_{1A}-C_{1A}-Hinge₁-(C_{H}2-C_{H}3)_{A} (I)
V_{1B}-C_{1B}-Hinge₂-(C_{H}2-C_{H}3)_{B}-L₁-V_{2A}-C_{2A}-Hinge₃ (II)
V_{2B}-C_{2B} (III)
wherein
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
L₁ is an amino acid linker; optionally wherein:
C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO:75;
Hinge₃ corresponds to the amino acid sequence of SEQ ID NO: 77;
L₁ corresponds to the amino acid sequence of SEQ ID NO: 76.

53. (canceled)

54. The method according to claim 29, wherein:
residue N297 of the Fc region or variant thereof according to EU numbering comprises a N-linked glycosylation; and/or
the all or part of the Fc region or variant thereof binds to a human CD16A (FcγRIII) polypeptide.

55. (canceled)

56. The method according to claim 52, comprising at least two polypeptide chains linked by at least one disulfide bridge; optionally wherein the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

57. (canceled)

58. The method according to claim 52, wherein:
V_{1A} is V_{L1} and V_{1B} is V_{H1}; and/or
V_{2A} is V_{H2} and V_{2B} is V_{L2}.

59. (canceled)

60. The method according to claim 29, wherein:
(a) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(b) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(d) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38;
(e) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;
(f) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(g) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(h) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(i) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or
(j) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

61. The method according to claim 29, wherein:
(a) V_{H1} and V_{L1} corresponds to the amino acid sequences of SEQ ID NO: 41 and 43 respectively or corresponds to the amino acid sequences of SEQ ID NO: 42 and 44 respectively; and/or
(b) V_{H2} and V_{L2} corresponds to
the amino acid sequences of SEQ ID NO: 45 and 53 respectively;
the amino acid sequences of SEQ ID NO: 46 and 54 respectively;
the amino acid sequences of SEQ ID NO: 47 and 55 respectively;
the amino acid sequences of SEQ ID NO: 48 and 56 respectively;
the amino acid sequences of SEQ ID NO: 49 and 57 respectively;
the amino acid sequences of SEQ ID NO: 50 and 58 respectively;
the amino acid sequences of SEQ ID NO: 51 and 59 respectively; or
the amino acid sequences of SEQ ID NO: 52 and 60 respectively, optionally wherein:
i. V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53:
ii. V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54;
iii. V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55;
iv. V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56;
v. V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57;
vi. V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58;
vii. V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59;
viii. V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60;
ix. V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53;
X. V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54:
xi. V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55;
xii. V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56;
xiii. V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57;
xiv. V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58;
xv. V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59;
xvi. V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60; and/or
polypeptide (I) consists of an amino acid sequence of SEQ ID NO: 64, polypeptide (II) consists of an amino acid sequence of SEQ ID NO: 65, and polypeptide (III) consists of an amino acid sequence of SEQ ID NO: 66.

62. (canceled)

63. (canceled)

64. The method of claim 1, wherein the hematological disease or disorder is a leukemia,
optionally wherein the leukemia is acute myeloid leukemia (AML), and/or
the AML is relapsed or refractory.

65-66. (canceled)

67. A method of treating or preventing a hematological disease or disorder in a subject in need thereof, the method comprising administering to the subject a Natural Killer (NK) cell and a binding protein comprising a first antigen binding domain with binding specificity to CD123 and a second antigen binding domain with binding specificity to NKp46,
wherein the first antigen binding domain comprises a heavy chain variable domain (VH1) and a light chain variable domain (VL1), wherein:
the VH1 comprises a complementary determining region (CDR)-H1, H2 and H3 corresponding to the amino acid sequences of SEQ ID NO: 1, 2, and 3; and
the VL1 comprises a CDR-L1, L2 and L3 corresponding to the amino acid sequences of SEQ ID NO: 7, 8, and 9; and
wherein the second antigen binding domain comprises a heavy chain variable domain (VH2) and a light chain variable domain (VL2), wherein:
the VH2 comprises a CDR-H1, H2, and H3 corresponding to the amino acid sequences of SEQ ID NO: 13, 14, and 15; and
the VL2 comprises a CDR-L1, L2, and L3 corresponding to the amino acid sequences of SEQ ID NO: 27, 28, and 29;
and wherein all or part of the immunoglobulin Fc region or variant thereof binds to a human Fc-γ receptor.

68. A method of treating or preventing a hematological disease or disorder in a subject in need thereof, the method comprising administering to the subject a Natural Killer (NK) cell and a binding protein comprising a first antigen binding domain with binding specificity to CD123, a second antigen binding domain with binding specificity to NKp46, and all or part of an immunoglobulin Fc region or variant thereof that binds to a human Fc-7 receptor, wherein the binding protein comprises:
a polypeptide (I) that comprises the amino acid sequence of SEQ ID NO: 64;
a polypeptide (II) that comprises the amino acid sequence of SEQ ID NO: 65; and
a polypeptide (III) that comprises the amino acid sequence of SEQ ID NO: 66.


SEQUENCE LISTING (116 sequences)

SEQ ID NO: 1 - protein, 10 aa, synthetic construct
GYSFTDYYMK

SEQ ID NO: 2 - protein, 10 aa, synthetic construct
DIIPSSGATF

SEQ ID NO: 3 - protein, 11 aa, synthetic construct
SHLLRASWFAY

SEQ ID NO: 4 - protein, 8 aa, synthetic construct
GFTFSHYN

SEQ ID NO: 5 - protein, 8 aa, synthetic construct
ITYDDHST

SEQ ID NO: 6 - protein, 10 aa, synthetic construct
ARLVNYAFAY

SEQ ID NO: 7 - protein, 17 aa, synthetic construct
ESSQSLLSSGNQKNYLT

SEQ ID NO: 8 - protein, 7 aa, synthetic construct
WASTRES

SEQ ID NO: 9 - protein, 9 aa, synthetic construct
QNDYSYPYT

SEQ ID NO: 10 - protein, 6 aa, synthetic construct
QTVGNN

SEQ ID NO: 11 - skipped

SEQ ID NO: 12 - protein, 8 aa, synthetic construct
QRMYNSPT

SEQ ID NO: 13 - protein, 5 aa, synthetic construct
DYVIN

SEQ ID NO: 14 - protein, 17 aa, synthetic construct
EIYPGSGTNYYNEKFKA

SEQ ID NO: 15 - protein, 11 aa, synthetic construct
RGRYGLYAMDY

SEQ ID NO: 16 - protein, 6 aa, synthetic construct
SDYAWN

SEQ ID NO: 17 - protein, 16 aa, synthetic construct
YITYSGSTSYNPSLES

SEQ ID NO: 18 - protein, 13 aa, synthetic construct
GGYYGSSWGVFAY

SEQ ID NO: 19 - protein, 5 aa, synthetic construct
EYTMH

SEQ ID NO: 20 - protein, 17 aa, synthetic construct
GISPNIGGTSYNQKFKG

SEQ ID NO: 21 - protein, 7 aa, synthetic construct
RGGSFDY

SEQ ID NO: 22 - protein, 5 aa, synthetic construct
SFTMH

SEQ ID NO: 23 - protein, 17 aa, synthetic construct
YINPSSGYTEYNQKFKD

SEQ ID NO: 24 - protein, 8 aa, synthetic construct
GSSRGFDY

SEQ ID NO: 25 - protein, 16 aa, synthetic construct
YITYSGSTNYNPSLKS

SEQ ID NO: 26 - protein, 11 aa, synthetic construct
CWDYALYAMDC

SEQ ID NO: 27 - protein, 11 aa, synthetic construct
RASQDISNYLN

SEQ ID NO: 28 - protein, 7 aa, synthetic construct
YTSRLHS

SEQ ID NO: 29 - protein, 9 aa, synthetic construct
QQGNTRPWT

SEQ ID NO: 30 - protein, 11 aa, synthetic construct
RVSENIYSYLA

SEQ ID NO: 31 - protein, 7 aa, synthetic construct
NAKTLAE

SEQ ID NO: 32 - protein, 9 aa, synthetic construct
QHHYGTPWT

SEQ ID NO: 33 - protein, 11 aa, synthetic construct
RASQSISDYLH

SEQ ID NO: 34 - protein, 7 aa, synthetic construct
YASQSIS

SEQ ID NO: 35 - protein, 9 aa, synthetic construct
QNGHSFPLT

SEQ ID NO: 36 - protein, 11 aa, synthetic construct
RASENIYSNLA

SEQ ID NO: 37 - protein, 7 aa, synthetic construct
AATNLAD

SEQ ID NO: 38 - protein, 9 aa, synthetic construct
QHFWGTPRT

SEQ ID NO: 39 - protein, 11 aa, synthetic construct
RTSENIYSYLA

SEQ ID NO: 40 - protein, 9 aa, synthetic construct
QHHYDTPLT

SEQ ID NO: 41 - protein, 120 aa, synthetic construct [sequence omitted]

SEQ ID NO: 42 - protein, 117 aa, synthetic construct
EVQLVESGGGLVQPGRSLKLSCAASGFTFSHYNMAWVRQAPKKGLEWVATITYDDHSTYYRDSVKGRFTISRDTAKSTLYLQMDSLRSEDTATYYCARLVNYAFAYWGQGTLVTVSS

SEQ ID NO: 43 - protein, 113 aa, synthetic construct [sequence omitted]

SEQ ID NO: 44 - protein, 106 aa, synthetic construct
NIVMTQSPKSMSISVGDRVTMNCKASQTVGNNIAWYQQKPGLSPQLLIDYASNRYTGVPNRFTGGGYGTDFILTINSVQAEDAAFYYCQRMYNSPTFGGGTKLELK

SEQ ID NO: 45 - protein, 120 aa, synthetic construct
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSS

SEQ ID NO: 46 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 47 - protein, 122 aa, synthetic construct
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 48 - protein, 116 aa, synthetic construct
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 49 - protein, 117 aa, synthetic construct
QVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSA

SEQ ID NO: 50 - protein, 122 aa, synthetic construct
QVQLQESGPGLVKPSQTLSLTCTVSGYSISSDYAWNWIRQPPGKGLEWIGYITYSGSTSYNPSLESRVTISRDTSKNQFSLKLSSVTAADTAVYYCARGGYYGSSWGVFAYWGQGTLVTVSS

SEQ ID NO: 51 - protein, 117 aa, synthetic construct
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSS

SEQ ID NO: 52 - protein, 120 aa, synthetic construct
QVQLQESGPGLVKPSQTLSLTCTVSGYSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 53 - protein, 107 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 54 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 55 - protein, 107 aa, synthetic construct
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 56 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 57 - protein, 107 aa, synthetic construct
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 58 - protein, 107 aa, synthetic construct
DIQMTQSPSSLSASVGDRVTITCRVSENIYSYLAWYQQKPGKAPKLLVYNAKTLAEGVPSRFSGSGSGTDFTLTISSLQPEDFATYYCQHHYGTPWTFGGGTKVEIK

SEQ ID NO: 59 - protein, 107 aa, synthetic construct
DIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIK

SEQ ID NO: 60 - protein, 107 aa, synthetic construct
DIQMTQSPSSLSASVGDRVTITCRTSENIYSYLAWCQQKPGKAPKLLIYNAKTLAEGVPSRFSGSGSGTDFTLTISSLQPEDFATYYCQHHYDTPLTFGQGTKLEIK

SEQ ID NO: 61 - protein, 467 aa, synthetic construct
MSVPTQVLGLLLLWLTDARCDIVMTQSPDSLAVSLGERATINCESSQSLLSSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 700 aa, synthetic construct
MEWSWVFLFFLSVTTGVHSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 63 - protein, 234 aa, synthetic construct
MSVPTQVLGLLLLWLTDARCDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 447 aa, synthetic construct
DIVMTQSPDSLAVSLGERATINCESSQSLLSSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 681 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 66 - protein, 214 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 107 aa, synthetic construct [sequence omitted]

SEQ ID NO: 68 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 69 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 72 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 10 aa, synthetic construct
DKTHTCPPCP

SEQ ID NO: 75 - protein, 15 aa, synthetic construct
EPKSCDKTHTCPPCP

SEQ ID NO: 76 - protein, 4 aa, synthetic construct
STGS

SEQ ID NO: 77 - protein, 10 aa, synthetic construct
EPKSCDKTHS

SEQ ID NO: 78 - protein, 9 aa, synthetic construct
EPKSCDKTH

SEQ ID NO: 79 - protein, 10 aa, synthetic construct
EPKSCDKTHT

SEQ ID NO: 80 - protein, 255 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQN

SEQ ID NO: 81 - protein, 262 aa, Macaca fascicularis
MSSTLRALLCLGLCLSQRISAPKQTLPKPIIRAESTYMVPKEKQATLCCQGSYGAVEYQLHFEGSLFAVERPKPPERINGVKFHIPDMNSRKAGRYSCIYRVGELWSERSDLLDLVVTEMYDTPTLSVHPGPEVTSGEKVTFYCRLDTATSMFLLLKEGRSRDVQRSYGKVQAEFPMGPVTTAHRGSYRCFGSYNNYAWSFPSEPVKLLVTGDIENTSLAPTDPTFPDSWDTCLLTRETGLQKDLALWDHTAQNDYKDDDDK

SEQ ID NO: 82 - protein, 287 aa, Homo sapiens
TKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWR

SEQ ID NO: 83 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 84 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 85 - protein, 441 aa, synthetic construct
EIVLTQSPATLSLSPGERATLSCRASQSVRSYLAWYQQKPGQAPRLLFSDASNRATGIPARFSGSGSGTDFTLTISSLEPEDFAVYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 678 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 87 - protein, 214 aa, synthetic construct; identical to SEQ ID NO: 66

SEQ ID NO: 88 - protein, 447 aa, synthetic construct; identical to SEQ ID NO: 64

SEQ ID NO: 89 - protein, 680 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVESGGGLVKPGGSLKLSCAASGFTFSDYGMHWVRQAPEKGLEWVAYISSGSSTIYYADTVKGRFTISRDNAKNTLFLQMTSLRSEDTAMYYCARGTTIFNYFEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 90 - protein, 220 aa, synthetic construct
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYEIPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 447 aa, synthetic construct; identical to SEQ ID NO: 64

SEQ ID NO: 92 - protein, 680 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 215 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 94 - protein, 447 aa, synthetic construct; identical to SEQ ID NO: 64

SEQ ID NO: 95 - protein, 679 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 216 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSTWTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 97 - protein, 214 aa, synthetic construct
EIVLTQSPATLSLSPGERATLSCRASQSVRSYLAWYQQKPGQAPRLLFSDASNRATGIPARFSGSGSGTDFTLTISSLEPEDFAVYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 447 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 6 aa, synthetic construct
QDISNY

SEQ ID NO: 100 - skipped

SEQ ID NO: 101 - protein, 10 aa, synthetic construct
QQGNTRPWTF

SEQ ID NO: 102 - protein, 8 aa, synthetic construct
GYTFSDYV

SEQ ID NO: 103 - protein, 8 aa, synthetic construct
IYPGSGTN

SEQ ID NO: 104 - protein, 13 aa, synthetic construct
ARRGRYGLYAMDY

SEQ ID NO: 105 - protein, 7 aa, synthetic construct
SQDISNY

SEQ ID NO: 106 - skipped

SEQ ID NO: 107 - protein, 6 aa, synthetic construct
GNTRPW

SEQ ID NO: 108 - protein, 7 aa, synthetic construct
GYTFSDY

SEQ ID NO: 109 - protein, 4 aa, synthetic construct
PGSG

SEQ ID NO: 110 - protein, 9 aa, synthetic construct
GRYGLYAMD

SEQ ID NO: 111 - protein, 8 aa, synthetic construct
ASQDISNY

SEQ ID NO: 112 - protein, 12 aa, synthetic construct
YTSRLHSGVPSR

SEQ ID NO: 113 - protein, 10 aa, synthetic construct
ASGYTFSDYV

SEQ ID NO: 114 - protein, 17 aa, synthetic construct
IYPGSGTNYYNEKFKAK

SEQ ID NO: 115 - protein, 10 aa, synthetic construct
RGRYGLYAMD

SEQ ID NO: 116 - protein, 9 aa, synthetic construct
QQGNTRPWT